(19) United States
(12) Patent Application Publication
(10) Pub. No.: US 2017/0101670 A1
(43) Pub. Date: Apr. 13, 2017

(54) METHOD FOR DETECTING RARE MUTATION

(71) Applicants: NATIONAL CANCER CENTER, Tokyo (JP); SYSMEX CORPORATION, Kobe-shi (JP)

(72) Inventors: Toshikazu USHIJIMA, Tokyo (JP); Satoshi YAMASHITA, Tokyo (JP)

(73) Assignees: NATIONAL CANCER CENTER, Tokyo (JP); SYSMEX CORPORATION, Kobe-shi (JP)

(21) Appl. No.: 15/287,121

(22) Filed: Oct. 6, 2016

(30) Foreign Application Priority Data
Oct. 7, 2015 (JP) 2015-199342

(51) Int. Cl. C12Q 1/68

(52) U.S. Cl. CPC C12Q 1/6827

(57) ABSTRACT

Disclosed is a method for detecting a rare mutation. The method comprises: preparing a sample comprising not more than 1,000 copies of template DNA; amplifying the template DNA to prepare a library, and analyzing a nucleotide sequence of the library; calculating a ratio of variants in a base at a predetermined position, from the analysis result; comparing the calculated ratio of variants with a predetermined cut-off value; and determining that the sample has a rare mutation in the base at the predetermined position when the calculated ratio of variants is not less than the predetermined cut-off value.

### CROSS REFERENCE TO RELATED APPLICATIONS

This application claims priority from prior Japanese Patent Application No. 2015-199342, filed on Oct. 7, 2015, entitled “Method for detecting rare mutation, detection device and computed program”, the entire contents of which are incorporated herein by reference.

### TECHNICAL FIELD

The present invention relates to a method for detecting a rare mutation.

### BACKGROUND

While it has been considered that the genome sequence of an individual is single, it has been revealed that there exists much genomic DNA having slightly different nucleotide sequences in an individual, based on the research using a next-generation sequencer. It is due to a generation of variation in the nucleotide sequence at a constant frequency during the development of reproductive cell, and a generation of variation in the nucleotide sequence at a constant frequency also during cell division and chromosomal replication. It is known that the variation of genome sequence generated as described above can be also one of the causes for onset of diseases.

Cancer is said to be developed by gradual generation of variation in the nucleotide sequence of oncogene and antioncogene. It is known that an individual cancer cell does not have a single genome sequence, but has various variations, by analyzing genomic DNA obtained from a tumor tissue by a next-generation sequencer. Shimizu T. et al., Accumulation of Somatic Mutations in TP53 in Gastric Epithelium With Helicobacter pylori Infection, Gastroenterology, 2014, vol. 147, No. 2, p. 407-417 discloses that whole exome sequencing and deep sequencing are performed for genomic DNA in a tumor tissue of stomach and a non-tumor tissue of stomach, and a somatic mutation is accumulated in various genes of gastric cancer tissue in which inflammation is caused.

When variation recognized at very low frequency in genomic DNA is detected by analysis of nucleotide sequence (hereinafter, also referred to as “sequencing”), a sufficient amount of genomic DNA is usually used as a template such that a genomic DNA molecule having the variation is surely contained in a sample.

For example, about 5 μg of a fragmented DNA is used as a template for DNA sequencing in Shimizu T. et al., Accumulation of Somatic Mutations in TP53 in Gastric Epithelium With Helicobacter pylori Infection, Gastroenterology, 2014, vol. 147, No. 2, p. 407-417. However, in the present technology, an error occurs at a predetermined frequency during nucleic acid amplification of a template DNA and during sequencing, thus variation derived from the error may be contained in the analyzed nucleotide sequence of the genomic DNA. Therefore, it is difficult to distinguish whether the variation of genomic DNA detected by sequencing is mutation or variation due to an error.

The present inventors have surprisingly found that it is possible to distinguish whether variation detected in a template DNA is mutation or variation due to an error, by sequencing using DNA in an amount much less than usual as a template. This finding has led to the completion of the present invention.

### SUMMARY

The scope of the present invention is defined solely by the appended claims, and is not affected to any degree by the statements within this summary.

The present invention provides a method for detecting a rare mutation. The method comprises the steps of: preparing a sample comprising not more than 1,000 copies of template DNA; amplifying the template DNA to prepare a library, and analyzing a nucleotide sequence of the library; calculating a ratio of variants in a base at a predetermined position, from the analysis result; comparing the calculated ratio of variants with a predetermined cut-off value; and determining that the sample has a rare mutation in the base at the predetermined position when the calculated ratio of variants is not less than the predetermined cut-off value.

The present invention further provides another method for detecting a rare mutation. The method comprises: dividing a sample comprising template DNA to prepare a plurality of aliquots each comprising not more than 1,000 copies of template DNA; amplifying the template DNA in a first aliquot to prepare a library, and analyzing a nucleotide sequence of the library; calculating a ratio of variants in a base at a predetermined position, from the analysis result; comparing the calculated ratio of variants with a predetermined cut-off value; executing the amplification and analysis step, the calculation step, and the comparison step using other aliquots; and determining that the sample has a rare mutation in the base at the predetermined position when the calculated ratio of variants in at least one of the aliquots is not less than the predetermined cut-off value.

The present invention provides another method for detecting a rare mutation. The method comprises the steps of: dividing a sample comprising template DNA to prepare a plurality of aliquots each comprising not more than 1,000 copies of template DNA; amplifying the template DNA in a first aliquot to prepare a library, and analyzing a nucleotide sequence of the library; calculating a ratio of variants in a base at a predetermined position, from the analysis result; comparing the calculated ratio of variants with a predetermined cut-off value; determining that the sample has a rare mutation in the base at the predetermined position when the calculated ratio of variants in the first aliquot is not less than the predetermined cut-off value; executing the amplification and analysis step, the calculation step, the comparison step and the determination step using a second aliquot when the calculated ratio of variants in the first aliquot is less than the predetermined cut-off value, and determining that the sample has a rare mutation in the base at the predetermined position when the calculated ratio of variants in the second aliquot is not less than the predetermined cut-off value.

### BRIEF DESCRIPTION OF THE DRAWINGS

FIG. 1A is a view showing a principle of conventional sequencing method using genomic DNA in a usual amount as a template;

FIG. 1B is a view showing a principle of a method for detecting a rare mutation of this embodiment;

FIG. 2 is a graph showing a frequency of somatic mutation induced by a mutagen;

FIG. 3A is a scatter diagram showing a frequency of variation in tissue mucosa DNA obtained from each patient group;

FIG. 3B is a ROC curve for distinguishing cancer patients, based on the frequency of variations of normal esophageal mucosa obtained from a healthy subject exposed to a risk factor for esophageal carcinogenesis and the frequency of variations of noncancerous esophageal mucosa obtained from a patient with esophagus squamous epithelium carcinoma;

FIG. 4 is a schematic diagram showing an example of a detection device;

FIG. 5 is a block diagram showing a hardware configuration of the detection device;

FIG. 6A is a flow chart of determination of the presence or absence of rare mutation using the detection device; and

FIG. 6B is a flow chart of determination of the presence or absence of rare mutation using the detection device.

### DETAILED DESCRIPTION OF THE PREFERRED EMBODIMENTS

### [1. Method for Detecting Rare Mutation]

In this embodiment, a “rare mutation” refers to variation of a base in a nucleic acid, generated in a living body, and intends to variation satisfying the following two conditions:
in a DNA molecule, the variation appears at a frequency of 1×10⁻³/base or less (i.e., a probability of 1 or less per 1,000 bases); and
in a sample containing a DNA molecule, the ratio of DNA molecule having the variation in a base at a predetermined position is 10% or less of the total number of DNA molecules in the sample.

The variation of the base may be any of substitution, insertion, and deletion, and is preferably substitution. In this embodiment, a base different from the original base at a predetermined position of a template DNA or a read described below is also called as “variant”. The variant may be derived from mutation, or may be derived from variation due to an error occurred in nucleic acid amplification or sequencing.

In this embodiment, SNP (single nucleotide polymorphism) is not included in rare mutations. It is because, while SNP is variation of genomic DNA recognized to appear at a frequency of 1×10⁻³/base or less, it is one type of genetic polymorphism in which a DNA molecule having SNP is recognized in a ratio of 50% or 100% (either or both of maternal allele and paternal allele), and is different from mutation, in a sample containing a DNA molecule of each individual.

A rare mutation may be generated in a living body due to various causes. For example, cells are exposed to a substance having a risk of causing mutagen or variation, whereby variation may be generated in DNA of a part of the cells. Such variation is also included in the “rare mutation” when the above conditions are satisfied. In diseases such as cancer, it is known that variation is likely to occur in DNA. In the canceration process, at the same time as mutation to be the main cause of disease (also referred to as driver mutation), mutation that does not become the cause of disease may be also generated, and such mutation is generally called as a passenger mutation. The passenger mutation in a non-cancerous tissue is generally said to appear at a frequency of 1×10⁻³/base or less randomly in various positions on DNA, and may be included in the “rare mutation”.

In the method for detecting a rare mutation of this embodiment (hereinafter simply also referred to as “detection method”), the lower limit of the frequency of rare mutations is theoretically not particularly limited. In this embodiment, as long as at least one rare mutation may be contained in not more than 1,000 copies of template DNA, it is possible to detect even a rare mutation recognized at a frequency of 1×10⁻⁴/base or less, 1×10⁻⁵/base or less, or 1×10⁻⁶/base or less. For example, in the case where a rare mutation with an appearance frequency of 1×10⁻⁶/base or less is detected, by analyzing a region of 10,000 bases for 100 copies of genomic DNA, one rare mutation may be theoretically contained in the analyzed region of 100 copies of genomic DNA (1×10⁻⁶×10000×100=1).

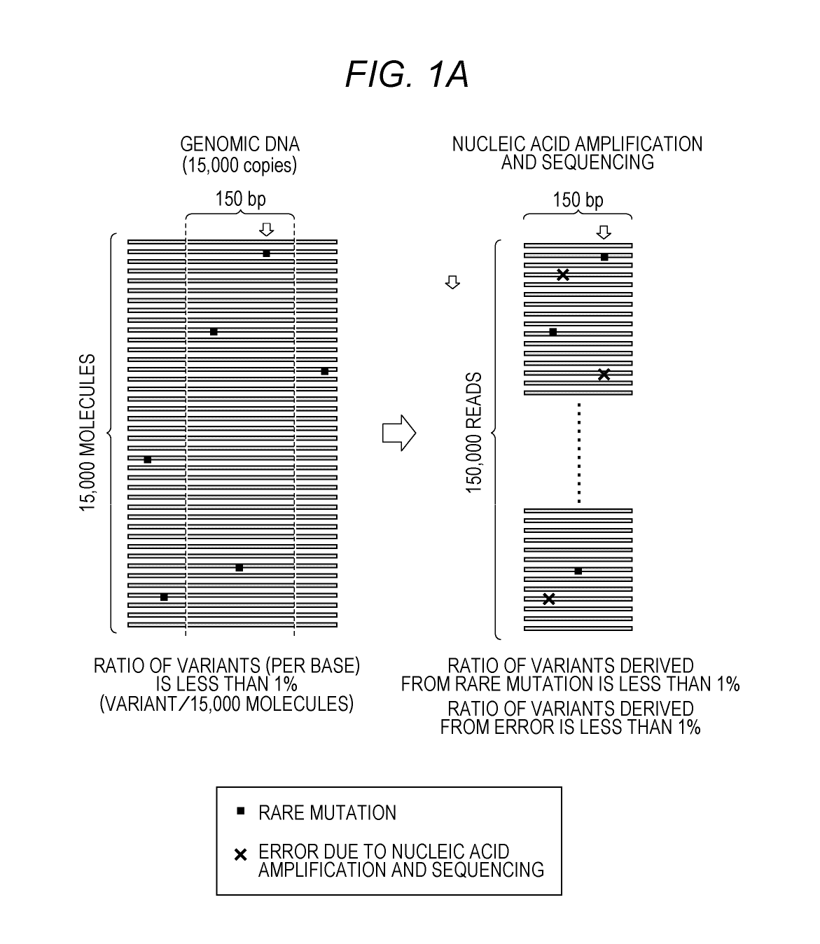

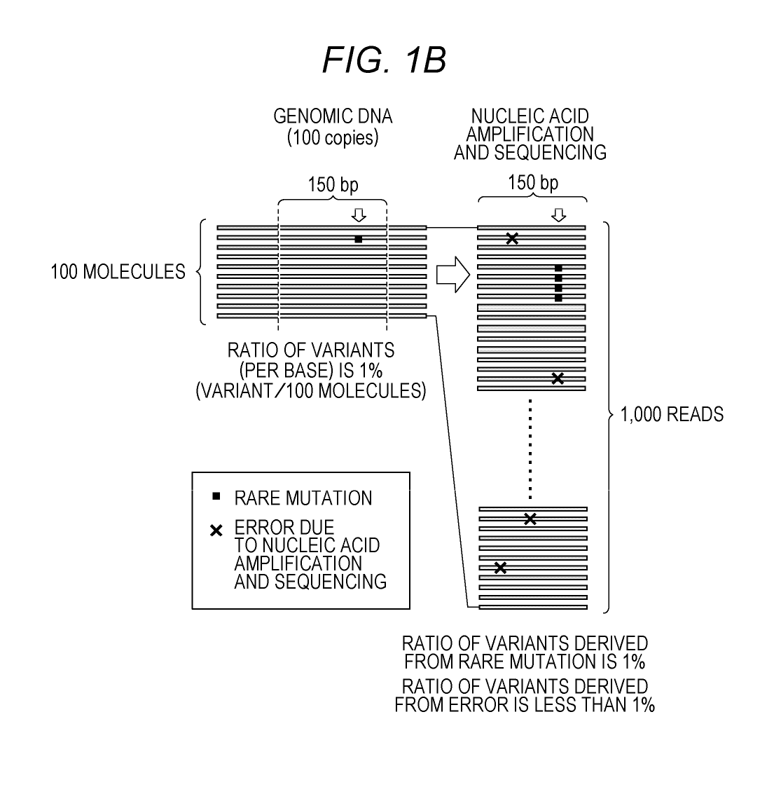

Hereinbelow, the principle of the detection method of this embodiment will be described with reference to FIGS. 1A and 1B. The following description is an example just for understanding the present disclosure, and does not limit the disclosure. First, a conventional sequencing method using genomic DNA in a usual amount as a template will be described with reference to FIG. 1A. The left side in FIG. 1A shows 15,000 copies of genomic DNA (corresponding to 50 ng) used as a template DNA. Each bar represents a genomic DNA molecule. The copy number of DNA herein has the same meaning as the number of DNA molecules. In the figure, “▪” represents a rare mutation, and the region sandwiched by two broken lines represents a predetermined region (150 bp) in which the nucleic acid is amplified (the same applies to FIG. 1B described later). In the conventional technology, when a desired region in genomic DNA is amplified by PCR, and a library prepared from amplicon (PCR product) is subjected to sequencing, 50 to 100 ng of genomic DNA is usually necessary as a template. In FIG. 1A, six rare mutations are contained in the 15,000 copies of genomic DNA, and three rare mutations are contained in the amplified region. The frequency of these rare mutations is 1.33×10⁻⁶/base in the amplified region (3/(150×15000)=1.33×10⁻⁶). The ratio of the number of genomic DNA molecules having a variant in the base at a predetermined position to the number of genomic DNA molecules in a sample is less than 1%. For example, in the base at a position indicated by an arrow, there is one variation in the 15,000 copies of genomic DNA, and therefore the ratio of variants is 6.66×10⁻³% ((1/15000)×100=6.66×10⁻³).

The right side in FIG. 1A shows an analysis result of the nucleotide sequence of a library prepared by PCR amplification of genomic DNA. Each bar represents a read. The “library” means an assembly of amplicon in which the nucleotide sequence is to be analyzed by a sequencer, and the “read” means a unit of amplicon in which the nucleotide sequence is analyzed by a sequencer. It shows a state that genomic DNA is amplified 10 times, and the obtained amplicon is all analyzed to obtain 150,000 reads. In the figure, “x” represents variation derived from an error due to nucleic acid amplification and sequencing (hereinafter, simply also referred to as “error”) (the same applies to FIG. 1B described later). The ratio of the number of reads containing a variant (hereinafter, simply also referred to as “the ratio of variants”) is calculated. The ratio of variants derived from the rare mutation is less than 1% similarly to the template DNA. The ratio of variants derived from the error is usually also less than 1%. Therefore, even when the variation in the template DNA is detected as the result of sequencing, it cannot distinguish whether this variation is derived from the rare mutation or derived from the error.

The above point will be more specifically described. With reference to FIG. 1A, when there is one rare mutation at the position indicated by an arrow in the genomic DNA, the number of reads having variation derived from this rare mutation is 10, due to nucleic acid amplification and sequencing. When the ratio of variants derived from the error is 0.1%, the number of reads having variation due to the error is 150 (150000×0.1/100=150). Therefore, the ratio of variants in the 150,000 reads is 0.106% ([(10+150)/150000]×100=0.106). On the other hand, when there is no rare mutation at the position indicated by an arrow in the genomic DNA, only variation derived from the error is contained in the reads. Accordingly, the ratio of variants in the 150,000 reads is 0.100% ((150/150000)×100=0.100). As described above, there is almost no difference in the ratio of variants between the case where there is a rare mutation (0.106%) and no rare mutation (0.100%) in the genomic DNA. Accordingly, in the conventional sequencing method that uses a usual amount of genomic DNA as a template, it cannot distinguish whether the detected variation is derived from the rare mutation or derived from the error.

The principle of the detection method of this embodiment will be described with reference to FIG. 1B. The left side in FIG. 1B shows 100 copies of genomic DNA (corresponding to 0.33 ng) used as a template DNA. In FIG. 1B, one rare mutation is contained in the 100 copies of genomic DNA. The frequency of this rare mutation is 6.66×10⁻⁵/base in the amplified region (1/(150×100)=6.66×10⁻⁵). For example, there is one variation in the 100 copies of genomic DNA in the base at a position indicated by an arrow, and therefore the ratio of the number of reads containing a variant is 1% ((1/100)×100=1). The right side in FIG. 1B shows reads. It shows a state that genomic DNA is amplified 10 times, and the obtained amplicon is all analyzed to obtain 1,000 reads. The ratio of variants derived from the rare mutation at this time is 1% similarly to the template DNA. On the other hand, the ratio of variants derived from the error is usually less than 1%. As described above, the ratio of variants derived from the rare mutation is higher than the ratio of variants derived from the error. Therefore, in the detection method of this embodiment, it can distinguish whether the variation detected by sequencing is derived from the rare mutation or derived from the error.

The above point will be more specifically described. With reference to FIG. 1B, when there is one rare mutation at the position indicated by an arrow in the genomic DNA, the number of reads having variation derived from this rare mutation is 10, due to nucleic acid amplification and sequencing. When the ratio of variants derived from the error is 0.1%, the number of reads having variation derived from the error is 1 (1000×0.1/100=1). Therefore, the ratio of variants in the 1,000 reads is 1.1% ([(10+1)/1000]×100=1.1). On the other hand, when there is no rare mutation at the position indicated by an arrow in the genomic DNA, only variation derived from the error is contained in the reads. Accordingly, the ratio of the number of reads having a variant in the 1,000 reads is 0.1% ((1/1000)×100=0.1). As described above, the difference in the ratio of variants between the case where there is a rare mutation (1.1%) and no rare mutation (0.1%) in the genomic DNA is increased. Accordingly, in the detection method of this embodiment, it is possible to distinguish whether the detected variation is derived from the rare mutation or derived from the error.

When the method of FIG. 1B is performed using a template DNA in which the presence or absence of a rare mutation is unknown, in each position on the reads obtained from the template DNA, the ratio of the number of the reads containing a base different from the original base (rare mutation or error) is calculated, and it is possible to determine which position the rare mutation is present. For example, in an amplification region of 150 bp, the base at position 1 is different from the original base at a ratio of about 1.1% in 1,000 reads, and when the base at any of positions 2 to 150 is different from the original base at a ratio of about 0.1%, it can be determined that the rare mutation is present in the base at position 1 in the amplification region.

According to the method shown in FIG. 1B, the number of template DNA molecules is small, so that stochastically, a variant derived from the rare mutation may not be contained in a sample. In this case, a site where the rare mutation is present may be specified by performing the method shown in FIG. 1B multiple times. For example, first, a sample containing a large amount of template DNA is divided into a plurality of aliquots. The sample is divided such that each aliquot contains not more than 1,000 copies of template DNA. Moreover, the method of FIG. 1B is performed on a first aliquot to detect a rare mutation. The method of FIG. 1B is performed on remaining respective aliquots as well. The sample is divided as described above, and the method shown in FIG. 1B is performed multiple times, whereby a rare mutation can be detected from a large amount of template DNA. More specifically, when 15,000 molecules of template DNA are all analyzed, 150 aliquots each containing 100 molecules of template DNA are prepared, and 150 analyses (the method of FIG. 1B) can be performed using each of a first aliquot to a one hundred and fiftieth aliquot. In this embodiment, a plurality of aliquots may be simultaneously analyzed, or each aliquot may be sequentially analyzed. For example, when a rare mutation is not detected in the analysis on the first aliquot, the analysis may be performed on the second aliquot. The number of aliquots is not particularly limited, as long as the number of template DNA molecules contained in each aliquot is 1,000 copies or less.

Each step of the detection method of this embodiment will be described below. In the detection method of this embodiment, first, a sample containing not more than 1,000 copies of template DNA is prepared.

The template DNA is not particularly limited, as long as it is DNA that may contain a rare mutation, and is preferably genomic DNA. The origin of the template DNA is not particularly limited, and may be any species of animals, plant, and microorganisms. Among them, genomic DNA of an organism in which the entire sequence of genomic DNA is analyzed is preferred, and human genomic DNA is particularly preferred. Human genomic DNA can be extracted, for example, from a biological sample. Examples of the biological sample include cells, tissues, body fluids, urine, feces, and the like. Examples of the body fluids include blood, serum, plasma, lymph, bone marrow fluid, ascites, amniotic fluid, semen, nipple discharge, and the like. DNA extracted from an FFPE (formalin-fixed paraffin-embedded) sample of tissue may be used.

The DNA extraction method is not particularly limited. When genomic DNA is extracted from a biological sample, it can be extracted by a known method in the art such as phenol/chloroform method. A commercially available DNA extraction kit and the like may be used. The fragmentation, size selection, terminal smoothing and the like of the extracted template DNA may be performed, as necessary.

In this embodiment, the lower limit of the copy number of the template DNA is at least 10 copies, preferably 30 copies, and more preferably 50 copies. The upper limit of the copy number of the template DNA is usually 1,000 copies, preferably 500 copies, and more preferably 200 copies. In this embodiment, when the copy number of the template DNA is in the range of 10 copies or more and 1,000 copies or less, it is possible to distinguish the ratio of variants derived from a rare mutation and the ratio of variants derived from an error due to nucleic acid amplification and sequencing. Particularly preferably, the copy number of the template DNA is 100 copies.

The means of adjusting the copy number of the template DNA in the sample to 1,000 copies or less is not particularly limited. It is known in the art that 1 ng of genomic DNA corresponds to 300 copies. Accordingly, the concentration of the genomic DNA extracted from the biological sample is measured by a spectrophotometer, and a sample containing not more than 1,000 copies, i.e., not more than 3.33 ng of the genomic DNA may be prepared by dilution based on the concentration. A predetermined gene in the template DNA may be quantitatively determined by real-time PCR, and the copy number of the template DNA may be determined from the quantitative result. As the predetermined gene to be quantitatively determined by real-time PCR, a gene present in any molecule of the template DNA is suitable. Examples of the gene include, in human genomic DNA, ALB, GAPDH, KCNA1, ARHGEF4, RAPGEFL1, and the like. Real-time PCR is particularly preferable since the accurate copy number of template DNA can be determined.

In the detection method of this embodiment, the template DNA contained in the sample is amplified to prepare a library, and sequencing of this library is performed.

The amplification of the template DNA is preferably performed by PCR-based method. A primer pair capable of amplifying a region to be analyzed in the template DNA is designed, and the template DNA is amplified by PCR method using this primer pair, whereby an amplicon can be obtained. The region to be analyzed is concentrated from the fragmented genomic DNA by sequence capture method, and an amplicon may be obtained using this region as template DNA.

The region to be analyzed can be determined from an arbitrary site in the template DNA. For example, in the case of genomic DNA, the region to be analyzed may be any of exon, intron, or a region containing both of them. Alternatively, the template DNA is previously subjected to sequencing, and based on the result, a region capable of ensuring a high number of reads or a region having less sequencing error may be selected as the region to be analyzed.

The lower limit of the length of the region to be analyzed (hereinafter, also referred to as “sequencing length”) is at least 1,000 bases, preferably 5,000 bases, and more preferably 10,000 bases, from the viewpoint of detecting mutation with a low appearance frequency. The upper limit of the sequencing length is theoretically not particularly limited. However, the longer the sequencing length is, the more the cost of sequencing increases. In this embodiment, the upper limit of the sequencing length is preferably 1,000,000 bases, and more preferably 100,000 bases.

The primer used in the amplification of the template DNA may have an addition sequence such as an adaptor sequence or a bar code sequence, a labeling substance or the like, depending on the kind of the sequencer to be used. The number of the primer pairs is determined by the desired sequencing length and the average length of the amplicon described below. The number of the primer pairs is counted as one pair by one forward primer and one reverse primer. The number of the primer pairs can be determined based on the following expression.

(Sequencing length)=(Average length of amplicon)×(Number of primer pairs)

When using a plurality of the primer pairs, it is preferred that multiplex PCR can be performed for these primer pairs. This makes it possible to simultaneously amplify a plurality of regions in the template DNA. In this case, it is preferred to add bar code sequences different each other to each primer pair. This makes it possible to distinguish the amplicon by each primer pair. A primer set for multiplex PCR attached to a commercially available kit such as an exome sequencing kit may be used.

The average length of the amplicon can be determined depending on the performance of the sequencer to be used, and should be usually at least 50 bp. The upper limit of the average length of the amplicon is theoretically not particularly limited. However, the length in which sequencing can be stably performed by the sequencer is preferred.

In the amplification of the template DNA by PCR, it is preferred to minimize the number of PCR cycles in the range where the number of reads necessary for sequencing is obtained, in order to suppress an error due to amplification. In this embodiment, the number of cycles should be determined, for example, from the range of 10 cycles or more and 25 cycles or less. It is considered in the art that, even when variation due to an error is introduced at a predetermined position of one molecule (amplified product) in PCR cycle, the probability that variation due to an error is simultaneously introduced also at the same position of other molecule is low. Accordingly, in the detection method of this embodiment, the ratio of variants derived from a rare mutation is higher than the ratio of variants derived from an error during nucleic acid amplification, so that both can be distinguished from each other.

A polymerase used in the amplification of the template DNA can be properly selected from known heat-resistant polymerases used in PCR. Among them, a heat-resistant polymerase suitable for multiplex PCR and having less PCR error is desirable. A buffer suitable for the selected polymerase should be used in the amplification reaction.

In this embodiment, the nucleotide sequence should be analyzed by a sequencing method known in the art for the library as described above. The sequencing method is not particularly limited, but the analysis by a next-generation sequencer is preferred. The “next-generation sequencer” is a term used as compared to a “first-generation sequencer” that is a sequencer by capillary electrophoresis using Sanger's method, and means a device that determines nucleotide sequences by treating several tens of millions to several hundred millions of DNA fragments simultaneously in parallel. In this embodiment, the next-generation sequencer is not particularly limited, but examples thereof include HiSeq 2500 (Illumina, Inc.), MiSeq (Illumina, Inc.), Ion Proton (Thermo Fisher Scientific Inc.), Ion PGM (Thermo Fisher Scientific Inc.), and the like.

In this embodiment, in order to enhance reliability of the determination result described below, it is desirable that the number of reads having variation derived from a rare mutation is at least 10 or more. For that purpose, the number of reads of sequencing is preferably 10 times or more the copy number of the template DNA, for a region to be amplified with each primer pair. On the other hand, the amplification efficiency may be sometimes different from each other in the amplification with a plurality of primer pairs, and thus the number of the amplicon may be different according to the amplified site. Therefore, the number of reads of sequencing also changes according to the amplified site. For example, in the analysis by Ion Proton sequencer (Thermo Fisher Scientific Inc.), it is known that, when the average number of reads is 5,000, the actual number of reads has dispersion of about 2,000 to 20,000 reads according to the amplified site. Therefore, in this embodiment, it is preferred that the average number of reads of sequencing is, for example, 25 times or more, and preferably 50 times or more the copy number of the template DNA. The number of reads can be digitally counted in numerical value by a next-generation sequencer. The average number of reads can be calculated by dividing all the number of reads by the number of primer pairs.

As for a species in which genome sequence has been already decoded, the genome sequence is generally available as a reference sequence in the art. In this embodiment, when the template DNA is derived from the species in which genome sequence has been already decoded, it is preferred to find variation by comparing the analyzed nucleotide sequence with the reference sequence. In the analysis by a next-generation sequencer, the presence or absence of variation can be detected in every read.

In this embodiment, the ratio of variants in a base at a predetermined position is calculated, based on the analysis result of the nucleotide sequences. As the predetermined position, a position is preferred where variation found by the comparison with the reference sequence is present. The ratio of variants in the base at this position is obtained, whereby whether the found variation is derived from a rare mutation or derived from an error can be determined. The ratio of variants in a base at a predetermined position is calculated by the following expression.

(Ratio of variants in base at predetermined position)=(Number of reads having variation in base at predetermined position)/(Number of reads containing base at predetermined position)

In the above expression, “Number of reads containing base at predetermined position” is a sum of the number of reads having variation in the base at the predetermined position and the number of reads having no variation in the base at the predetermined position. As shown in FIG. 1B, since the appearance frequency of the rare mutation is low, there exist template DNA having the rare mutation and template DNA having no rare mutation, in the template DNA molecules in the sample. An error due to nucleic acid amplification and sequencing also randomly occurs at a low frequency. Therefore, in the reads, a read having variation in the base at the predetermined position and a read having no variation in the base at the predetermined position exist.

In this embodiment, the ratio of variants is preferably calculated for each one base in the region to be analyzed. In the region to be analyzed, when a plurality of variations is present in the positions being different from each other, the ratio of variants is calculated for the base at the position where each variation is present.

In this embodiment, the calculated ratio of variants is compared with a predetermined cut-off value, and whether or not the sample has a rare mutation in the base at the predetermined position is determined, based on the result. Specifically, when the calculated ratio of variants is not less than the predetermined cut-off value, it is determined that the sample has a rare mutation in the base at the predetermined position. On the other hand, when the calculated ratio of variants is lower than the predetermined cut-off value, it is determined that the sample has no rare mutation in the base at the predetermined position. When it is determined that the sample has no rare mutation in the base at the predetermined position, it may be determined that the variation in the base at this position is derived from an error.

In this embodiment, the predetermined cut-off value may be the ratio of variants derived from an error. The distribution of an error due to nucleic acid amplification and sequencing is considered to follow the Poisson distribution that is a distribution of random events at a low frequency. Therefore, the predetermined cut-off value can be determined from the Poisson probability obtained from the Poisson distribution based on the Phred scores of the analyzed nucleotide sequence and the number of reads. The predetermined cut-off value may be set for each one base in the region to be analyzed, but it is preferred to set a single cut-off value based on the average value of the Phred scores of the analyzed nucleotide sequence and the average number of reads because of convenience.

The “Phred” refers to a base calling program used in a DNA sequencer, and is known in the art. Phred enables to execute base calling (determination of base) from the trace data (graph image such as waveform data of signals obtained from sequencing reaction) acquired by a DNA sequencer. At this time, a Phred score (also called as “Phred quality score”) is calculated for each designated base. The Phred score is an index representing accuracy of the nucleotide sequence analyzed by a sequencer, and widely spread in the art. The relationship between the Phred score (or the average value thereof) and the frequency of errors in the analyzed nucleotide sequence is represented by the following expression.

(Frequency of errors)=10^{-a/10}(/base)

wherein a is a Phred score or an average value thereof.

For example, when the Phred score of one base is 20, the frequency of errors in the base is 1×10⁻²/base, and when the Phred score is 30, the frequency of errors in the base is 1×10⁻³/base. The average value of the Phred score can represent the frequency of errors in the analyzed nucleotide sequence. For example, when the average value of the Phred score is 20, an error occurs once per 100 bases (1×10⁻²/base), and when the average value of the Phred score is 30, an error occurs once per 1,000 bases (1×10⁻³/base).

The Phred score of each base is automatically calculated by a next-generation sequencer. The average value of the Phred score can be calculated by dividing the sum of the Phred scores of the analyzed nucleotide sequence by the number of the analyzed bases. The Phred score differs depending on the sequencer to be used. For example, in the case of Ion Proton sequencer used in the examples, the average value of the Phred scores of the analyzed nucleotide sequence is about 25.

In this embodiment, it is preferred to set, as the predetermined cut-off value, the ratio of variants when the expected value of the number of variations due to an error in the sequencing length is 1 or less. The ratio of such variants is calculated from the Poisson probability obtained from the Poisson distribution based on the average value of the Phred scores of the analyzed nucleotide sequence and the average number of reads, and the sequencing length. The calculation example of the predetermined cut-off value will be described below.

### Calculation Example of Predetermined Cut-Off Value

As for 100 copies of genomic DNA, the nucleotide sequence was analyzed by a next-generation sequencer. In this analysis, the sequencing length was 10,000 bases, the average value of the Phred score was 30, and the average number of reads was 5,000. The frequency of errors in the sequencing length is 1×10⁻³/base (10^{-30/10}=1×10⁻³) since the average value of the Phred score is 30. Since the average number of reads is 5,000, the average of the Poisson distribution is 5 (5000×1×10⁻³=5). That is, the number of reads having variation due to an error per 5,000 reads is 5 in average. The relationship of the average of the Poisson distribution, the average number of reads and the average value of the Phred scores are represented by the following expression.

(Average of Poisson distribution)=(Average number of reads)×10^{-a/10}

wherein a is an average value of the Phred scores.

Subsequently, the distribution of probability (Poisson distribution) will be determined when the number of reads (the number of events) having variation due to an error per 5,000 reads is k. The probability P(k) is calculated by the following expression (0!=1).

P(k)=e⁻λ(λ^{k}/k!)

wherein λ is the average of the Poisson distribution, and k is the number of events.

The Poisson distribution may be calculated using spreadsheet software capable of performing statistical processing. Examples of such spreadsheet software include Excel (registered trademark) (Microsoft Corporation) and the like. Specifically, a table of the Poisson probability is prepared by Excel (registered trademark) when the number of events is 0 to 50, with an average of the Poisson distribution of 5, the number of events of 0 to 50, and a functional form of FALSE. In this example, the upper limit of the number of events is the average number of reads itself (i.e., 5,000). However, the frequency of occurrence of error is low, and therefore the Poisson probability may be usually calculated by setting the upper limit of the number of events to 1/50 or less the average number of reads. Moreover, the expected value of the number of variations due to an error in the sequencing length was calculated based on the following expression.

(Expected value of number of variations due to error)=(Sequencing length)×(Poisson probability)

The number of events (the number of reads having variation) was 0 to 2 and 16 to 50 when the calculated expected value was 1 or less, namely, the number of variations due to an error in 10,000 bases was 1 or less. The expected value when the number of events was 0 to 2 was apparently 1 or less, but it is highly likely to underestimate the occurrence of error. Herein, 16 was used as the number of events when the expected value was 1 or less, for calculating the lowest predetermined cut-off value. P(16)=4.91×10⁻⁵, and the expected value is 0.491 (4.91×10⁻⁵×10000=0.491). The ratio of variants derived from an error at this time is 0.32%, since 16 errors are present in the 5,000 reads ((16/5000)×100=0.32). Accordingly, 0.32% can be set as the predetermined cut-off value.

In the case where the Phred score is a relatively low value (e.g., 27 or less), the number of events (referred to as “k′”) when the calculated expected value is 1 or less can take the low value (or group of low values) and the high value (or group of high values), in 0 or more, as the example described above. When using a low value or a value selected from the group of low values as k′, the ratio of variants derived from an error is underestimated. Accordingly, in this embodiment, it is desirable to use a high value or a value selected from the group of high values as k′. When the lowest value among the group of high values is used as k′, the lowest predetermined cut-off value can be calculated.

When the average number of reads and the average value of Phred score obtained from the used next-generation sequencer are stable between analyses to some extent, the predetermined cut-off value may not be calculated each time the detection method of this embodiment is carried out. That is, a fixed value may be used as the predetermined cut-off value. The fixed value can be calculated from the average number of reads and the average value of Phred score empirically obtained by the used next-generation sequencer as described above.

As described above, in this embodiment, when the ratio of variants in the base at the predetermined position is not less than the predetermined cut-off value, it is determined that the sample has a rare mutation in the base at the predetermined position. However, when the ratio of variants in the base at the predetermined position is too high, this variation in the base at the predetermined position may not be a rare mutation. For example, the variation in the template DNA is SNP, the ratio of variants in the base at the position of SNP is theoretically 50% or 100%. SNP is one type of genetic polymorphism, and is desirably distinguished from the rare mutation to be detected in this disclosure. In this embodiment, the ratio of variants in the base at the predetermined position is preferably 10% or less.

### [2. Rare Mutation Detection Device and Computer Program]

The scope of this disclosure also includes a rare mutation detection device (hereinafter, also referred to as “detection device”). The scope of this disclosure also includes a computer program for enabling a computer to execute detection of a rare mutation (hereinafter, also referred to as “computer program”).

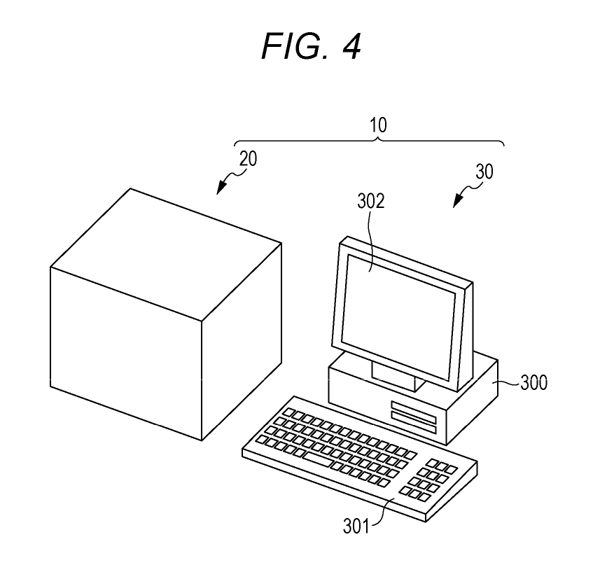

Hereinbelow, an example of the detection device will be described with reference to a figure. However, this embodiment is not limited only to a configuration shown in this example. FIG. 4 is a schematic diagram of a detection system of rare mutation. A detection system 10 of rare mutation shown in FIG. 4 includes a sequencer 20 and a detection device 30 connected to the sequencer 20. The detection device 30 is shown in FIG. 4 as a computer system including a computer body 300, an input unit 301 and a display unit 302, but is not limited to this configuration. The detection device 30 may be an instrument separated from the sequencer 20 as shown in FIG. 4, or may be an instrument including the sequencer 20. In the latter case, the detection device 30 may be used as the detection system 10 by itself. The sequencer 20 is preferably a next-generation sequencer. The computer program of this embodiment may be loaded into a commercially available next-generation sequencer.

When a library prepared by a nucleic acid amplification reaction using a sample containing not more than 1,000 copies of template DNA is set in the sequencer 20, the sequencer 20 executes analysis of the nucleotide sequence of the library, and acquires information such as the analyzed nucleotide sequence, and the Phred score, number of reads and sequencing length of each base, and the obtained various information is transmitted to the detection device 30 as analysis data. A format of the analysis data is not particularly limited, and may be a format corresponding to the used sequencer. Examples of such a format include FASTA format and the like.

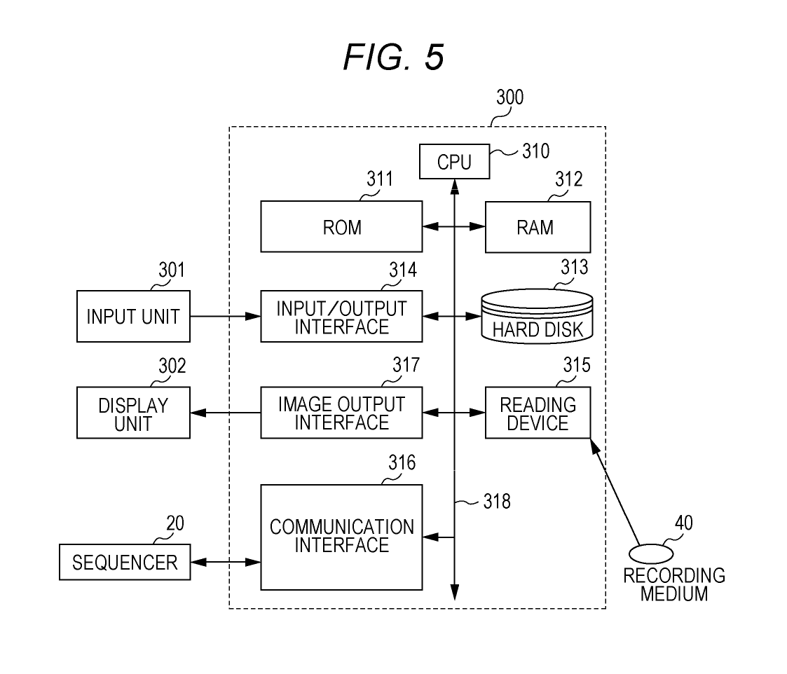

The detection device 30 receives the analysis data from the sequencer 20. A processor (CPU) of the detection device 30 executes a computer program for detection of a rare mutation, the program being installed on hard disk 313 (refer to FIG. 5), based on the analysis data.

With reference to FIG. 5, the computer body 300 includes a CPU (Central Processing Unit) 310, a ROM (Read Only Memory) 311, a RAM (Random Access Memory) 312, a hard disk 313, an input/output interface 314, a reading device 315, a communication interface 316, and an image output interface 317. The CPU 310, the ROM 311, the RAM 312, the hard disk 313, the input/output interface 314, the reading device 315, the communication interface 316 and the image output interface 317 are data-communicatively connected by a bus 318. The computer body 300 is communicatively connected to the sequencer 20 via the communication interface 316. The computer body 300 transmits and receives data with the sequencer 20.

The CPU 310 can execute programs stored in the ROM 311 or the hard disk 313 and programs loaded in the RAM 312. The CPU 310 calculates the ratio of variants in a base at a predetermined position, and reads out a predetermined cut-off value stored in the ROM 311 or the hard disk 313, to determine the presence or absence of a rare mutation in the base at the predetermined position. The CPU 310 outputs a determination result and allows the display unit 302 to display the result.

The ROM 311 is configured by mask ROM, PROM, EPROM, EEPROM, or the like. The ROM 311 records the computer programs to be executed by the CPU 310 and the data used in executing the computer programs as described above. The ROM 311 may record the predetermined cut-off value. The ROM 311 may record the expression for calculating the average number of reads, the expression for calculating the average value of Phred scores, the expression for calculating the Poisson distribution, the reference sequence, and the like.

The RAM 312 is configured by SRAM, DRAM, or the like. The RAM 312 is used to read out the programs recorded on the ROM 311 and the hard disk 313. In executing these programs, the RAM 312 is used as a work region of the CPU 310.

The hard disk 313 is installed with programs to be executed by the CPU 310 such as operating system and application program (computer program of this embodiment), as well as the data used in executing the program. The hard disk 313 may record the predetermined cut-off value. The hard disk 313 may record the expression for calculating the average number of reads, the expression for calculating the average value of Phred scores, the expression for calculating the Poisson distribution, the reference sequence, and the like.

The input/output interface 314 is configured, for example, by serial interface such as USB, IEEE 1394 or RS-232C; parallel interface such as SCSI, IDE or IEEE1284; and an analog interface including D/A or A/D converter. The input/output interface 314 is connected to the input unit 301 including a keyboard and a mouse. An operator can input various commands and data into the computer body 300 by the input unit 301.

The reading device 315 is configured by a flexible disk drive, CD-ROM drive, DVD-ROM drive, or the like. The reading device 315 can read programs or data recorded on a portable recording medium 40.

The communication interface 316 is, for example, Ethernet (registered trademark) interface, or the like. The computer body 300 can transmit print data to a printer by the communication interface 316.

The image output interface 317 is connected to the display unit 302 configured by LCD, CRT, or the like. This makes it possible for the display unit 302 to output a video signal corresponding image data provided from the CPU 310. The display unit 302 displays an image (screen) according to the input video signal.

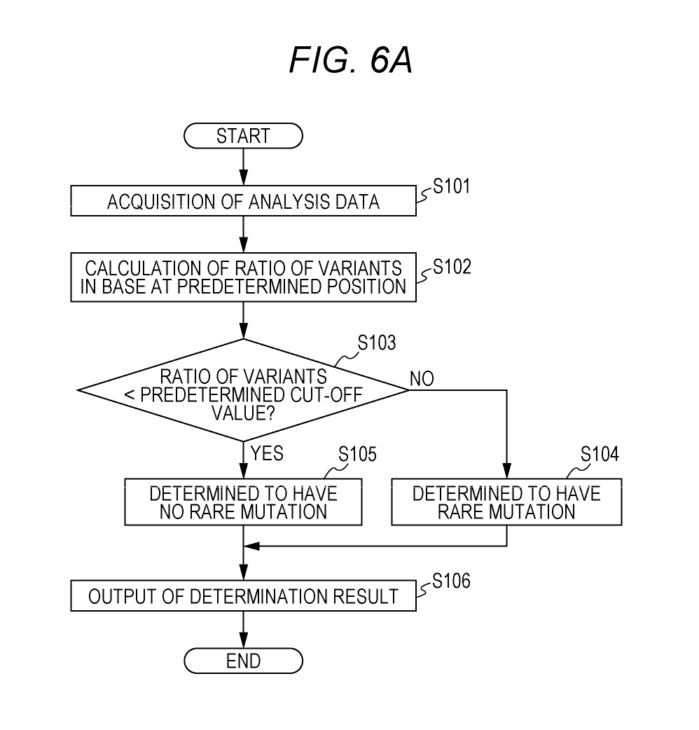

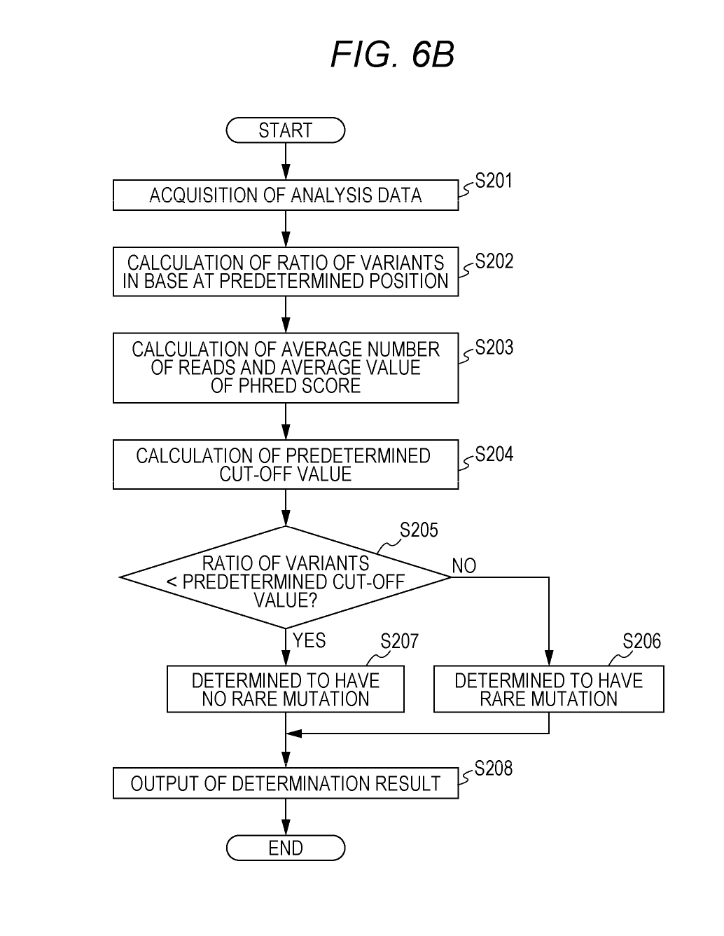

With reference to FIG. 6A, a determination flow of the presence or absence of a rare mutation executed by the detection device 30 will be described. The case will be described as an example where the ratio of variants in the base at the predetermined position is calculated from the analysis data acquired from the sequencer 20 that is a next-generation sequencer, and a determination is performed using the ratio of variants and the predetermined cut-off value previously stored in the memory. However, this embodiment is not limited only to this example.

In Step S101, the CPU 310 acquires analysis data from the sequencer 20, and stores the analyzed nucleotide sequence and the number of reads in the hard disk 313. In Step S102, the CPU 310 calculates the ratio of variants in the base at the predetermined position based on the stored number of reads, and stores it in the hard disk 313. The base at the predetermined position is preferably at a position where variation is present with respect to the reference sequence. The calculation of the ratio of variants is the same as that stated in the detection method of this embodiment. In Step S103, the CPU 310 compares the calculated ratio of variants with the predetermined cut-off value stored in the hard disk 313. When the calculated ratio of variants is equal to or higher than the predetermined cut-off value, the processing proceeds to Step S104, and the determination result showing that a rare mutation is present in the base at the predetermined position is stored in the hard disk 313. On the other hand, when the calculated ratio of variants is lower than the predetermined cut-off value, the processing proceeds to Step S105, and the determination result showing that a rare mutation is absent in the base at the predetermined position is stored in the hard disk 313. In Step S106, the CPU 310 outputs a determination result, allows the display unit 302 to display, and allows a printer to print the result.

With reference to FIG. 6B, a determination flow of the presence or absence of a rare mutation will be described. The case will be described as an example where the ratio of variants in the base at the predetermined position and the predetermined cut-off value are calculated from the analysis data acquired from the sequencer 20 that is a next-generation sequencer, and a determination is performed using the calculated ratio of variants and the calculated predetermined cut-off value. However, this embodiment is not limited only to this example.

In Step S201, the CPU 310 acquires analysis data from the sequencer 20, and stores the analyzed nucleotide sequence, the number of reads and the Phred score of each base in the hard disk 313. In Step S202, in the same manner as in Step S102 described above, the ratio of variants in the base at the predetermined position is calculated based on the stored number of reads, and is stored in the hard disk 313. In Step S203, the CPU 310 calculates the average number of reads based on the stored number of reads, calculates the average value of the Phred scores based on the stored Phred scores, and stores these values in the hard disk 313. The calculation of these values is the same as that stated in the detection method of this embodiment. In Step S204, the CPU 310 calculates the ratio of variants when the expected value of the number of variations due to an error in the sequencing length is 1 or less, based on the stored average number of reads and average value of the Phred scores, and stores this value in the hard disk 313 as the predetermined cut-off value. The calculation of this predetermined cut-off value is the same as that stated in the detection method of this embodiment. In Step S205, the CPU 310 compares the calculated ratio of variants with the calculated predetermined cut-off value. When the calculated ratio of variants is equal to or higher than the predetermined cut-off value, the processing proceeds to Step S206, and the determination result showing that a rare mutation is present in the base at the predetermined position is stored in the hard disk 313. On the other hand, when the calculated ratio of variants is lower than the predetermined cut-off value, the processing proceeds to Step S207, and the determination result showing that a rare mutation is absent in the base at the predetermined position is stored in the hard disk 313. In Step S208, the CPU 310 outputs a determination result, allows the display unit 302 to display, and allows a printer to print the result.

When dividing a sample to prepare a plurality of aliquots, the preparation of the plurality of aliquots can be also automatically performed by a device. When the detection method of this embodiment is performed using a first aliquot, and a rare mutation is not detected, the detection using a second aliquot may be automatically performed. The sequencer 20 and the detection device 30 may be configured such that the analysis of aliquots is automatically repeated until a rare mutation is detected.

This disclosure will be described in more detail by examples hereinbelow. However, this disclosure is not limited to these examples.

### EXAMPLES

### Example 1

In Example 1, N-nitroso-N-methylurea (hereinafter referred to as “MNU”) that was a mutagen was administered to cultured cells, to induce a point mutation of genomic DNA. Then, mutation was detected by the detection method of this embodiment, and the appearance frequency of the mutation was calculated. This analysis was independently performed three times.

### (1) Administration of Cells and Mutagen

Human TK6 lymphoblasts (hereinafter, referred to as “TK6 cell”) were obtained from American Type Culture Collection. On day 0, 1×10⁵ cells of TK6 cells were seeded on a 10 cm plate. On day 1, the TK6 cells were exposed to MNU (Sigma) in a concentration of 0, 0.1, 0.3, 1, 3, 10 or 30 μM for 24 hours. On day 7, the number of cells was counted, and the cells were collected. Then, genomic DNA was extracted by phenol/chloroform method.

### (2) Quantitative Determination of Copy Number of Genomic DNA

The copy number of the extracted genomic DNA was determined quantitatively by real-time PCR using SYBR (registered trademark) green I (BioWhittaker Molecular Applications) and iCycler Thermal Cycler (Bio-Rad Laboratories, Inc.). Genes to be measured and sequences of the primer are shown in Table 1. In the table, “F” means a forward primer, and “R” means a reverse primer. Each sample was measured using three kinds of primers. The average value of three copy numbers obtained above was defined as the DNA copy number of the sample.

TABLE 1

| Gene | Chromo- | | | Sequence | Length | Annealing |
|---|---|---|---|---|---|---|
| symbol | some | Genomic region | Primer sequence | number | (bp) | temperature (° C.) |
| RAPGEFL1 | 17q21.1 | 38348396-38348530 | F: ATCCGAGGCTCCCATGTAAC | 1 | 135 | 57 |
| | | | R: GCCAAACCCACTCACCGTCA | 2 | | |
| ARHGEF4 | 2q22 | 131784295-131784395 | F: AATGTCTCGTAATGCCAATC | 3 | 101 | 56 |
| | | | R: CCTAGGCACACCAAATAGTT | 4 | | |
| ALB | 4q13.3 | 74274349-74274498 | F: TCTTCGTGAAACCTATGGTGA | 5 | 150 | 60 |
| | | | R: TCATGAAAAGCAGTGCACA | 6 | | |

### (3) Detection of Rare Mutation

A sample containing 100 copies of genomic DNA was prepared, based on the measurement result of the copy number. A library for sequencing was prepared by amplification with multiplex PCR, using 100 copies of genomic DNA in the sample as a template. For the preparation of this library, Ion AmpliSeq Library Kit 2.0 (Thermo Fisher Scientific Inc.) was used. Specific operation was performed in accordance with the instruction attached to the kit. In multiplex PCR, 291 primer pairs (sequence numbers 7 to 588: sequences represented by add sequence numbers are each a sequence of a forward primer, and sequences represented by even sequence numbers are each a sequence of a reverse primer) were used. This made 291 regions in 55 cancer-related genes on the genomic DNA amplified at the same time. These primer pairs cover 48,587 bp. To the amplicon in the library is added a bar code sequence corresponding to each sample by the kit. The resulting library was subjected to sequencing by Ion PI Chip and Ion Proton sequencer (Thermo Fisher Scientific Inc.). The acquired nucleotide sequence data was mapped to the human reference genome hg19 using Ion Suite 4.0 (Thermo Fisher Scientific Inc.) to determine a nucleotide sequence. The average number of reads of sequencing was 5,000. Among the analyzed 48,587 bases, 15,724 bases were selected. It is because, in this selected region, the average number of reads in independent three times of analysis is 2,500 or more in untreated TK6 cells, and this selected region does not contain variation with a ratio of variants of 0.2% or more in the untreated TK6 cells.

When there is one variation in the 100 copies of genomic DNA, the ratio of variants is theoretically 1%. This ratio is considered to be higher than the ratio of variants derived from an error due to PCR and sequencing described above. The ratio of variants derived from an error was calculated as follows. The average value of the Phred scores of the nucleotide sequence analyzed by Ion Proton sequencer was 25. Accordingly, the frequency of errors is 3.16×10⁻³/base (10^{-25/10}=3.16×10⁻³). Since the average number of reads is 5,000, the average of the Poisson distribution is 15.8 (5000×3.16×10⁻³=15.8). Moreover, using the number of reads having an error in the 5,000 reads as the number of events of the Poisson probability, a table of the Poisson probability was formed by spreadsheet program Excel (registered trademark) (Microsoft) (average of the Poisson distribution: 15.8, the number of events: 0 to 60, functional form: FALSE). Then, the expected value of the number of variations due to the error in the region selected above was calculated from the product of the Poisson probability in each of the number of events and the length (15,724 bases) of the selected region. The number of events (the number of reads having variation) was 33 when the resulting expected value was 1 or less, namely, when the number of variations due to the error in the 15,724 bases was 1 or less. In this case, the ratio of variants derived from the error is 0.66% ((33/5000)×100=0.66). Accordingly, in the analyzed nucleotide sequence, variation with a ratio of variants of higher than 0.66% is considered to be a somatic mutation induced by MNU, not variation due to the error. In Example 1, variation with a ratio of variants of 0.8 to 10% was detected as a somatic mutation induced by MNU. Then, the frequency of the detected variations was calculated as the number of variations in 1,572,400 bases (15,724 bases×100 copies).

### (4) Result

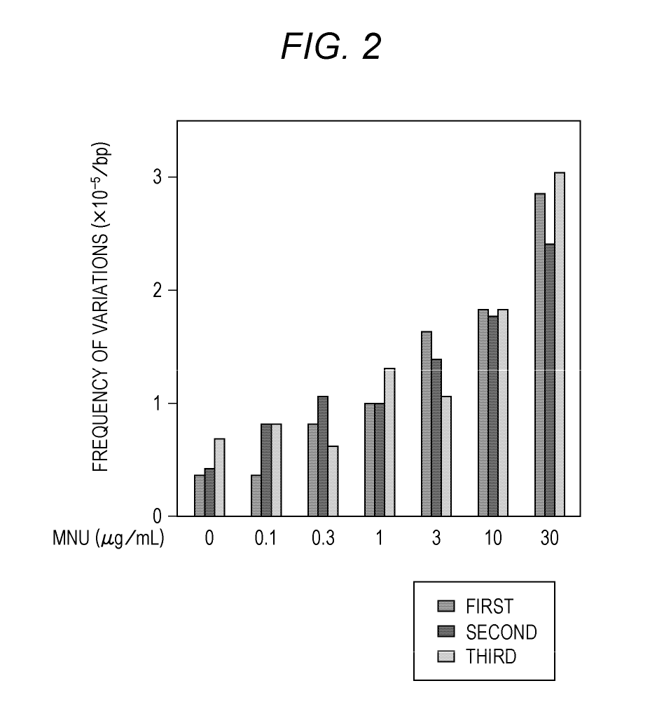

The result of three times of analysis independently performed is shown in FIG. 2. In FIG. 2, the horizontal axis denotes the concentration of MNU, and the vertical axis denotes the appearance frequency of point mutation. As shown in FIG. 2, it was found that there is a correlation between the administration amount of MNU and the accumulation of mutations. Despite that the frequency of mutations induced by MNU is very low, it was shown that mutation can be detected by using the detection method of Example 1.

### Example 2

In Example 2, using esophageal mucosa collected from a donor as a specimen, a point mutation in those genomic DNA was detected by the detection method of this embodiment, and the appearance frequency was calculated.

### (1) Tissue Specimen

291 specimens of esophageal mucosa were collected from adults who underwent cancer screening inspection between September, 2008 and April, 2013, using an endoscope. From a donor of each specimen, history information regarding risk factors for esophageal carcinogenesis of alcohol drinking, betel quid chewing, and cigarette smoking (hereinafter also referred to as “ABC”) was obtained by interview (refer to Y. C. Lee et al., Cancer Prev Res (Phila), 2011, vol. 4, p. 1982 to 1992). 93 specimens were classified into the following three groups according to the risk of cancer.

Group 1: Normal esophageal mucosa obtained from healthy subjects not exposed to ABC (30 specimens)

Group 2: Normal esophageal mucosa obtained from healthy subjects exposed to ABC (32 specimens)

Group 3: Noncancerous esophageal mucosa obtained from patients with esophagus squamous epithelium carcinoma (31 specimens)

### (2) Extraction and Quantitative Determination of Copy Number of Genomic DNA

Genomic DNA was extracted from each specimen by phenol/chloroform method. As to the resulting genomic DNA, the copy number was quantitatively determined in the same manner as in Example 1, and a sample containing 100 copies of genomic DNA was prepared.

### (3) Detection of Rare Mutation

As to the sample containing 100 copies of genomic DNA prepared from each specimen, a library for sequencing was prepared in the same manner as in Example 1, and subjected to sequencing by Ion PI Chip and Ion Proton sequencer (Thermo Fisher Scientific Inc.). Then, the variation in the genomic DNA was detected in distinction from the variation derived from an error, and the appearance frequency of variations was calculated in the same manner as in Example 1.

### (4) Result

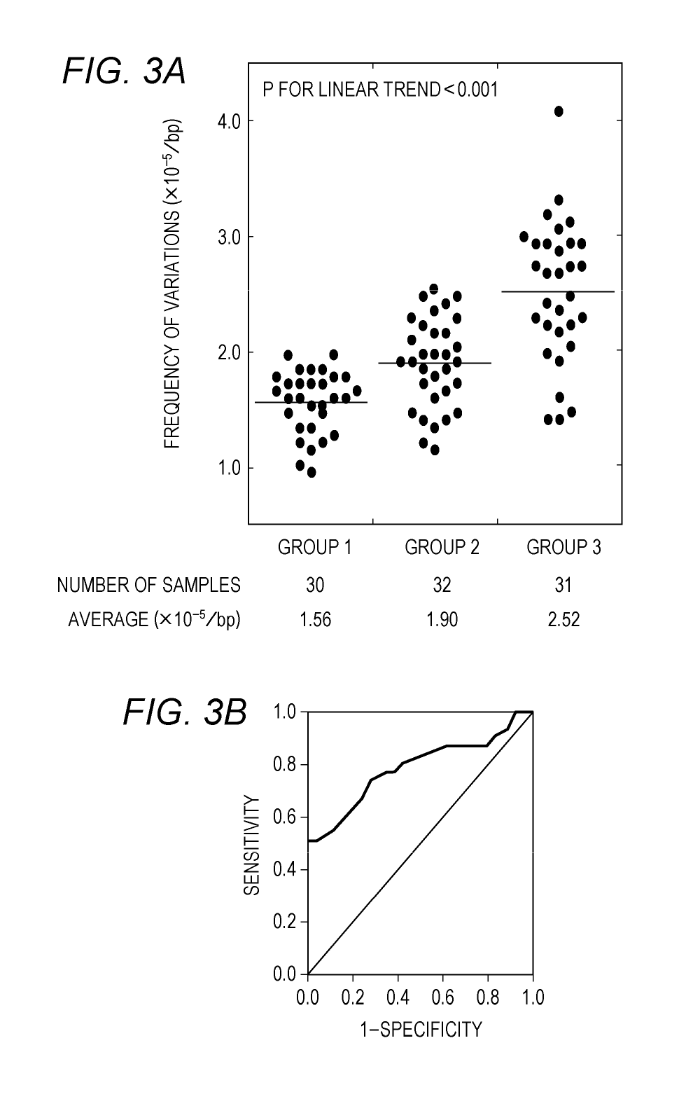

The appearance frequency of variations in each group is shown in FIG. 3A. In FIG. 3A, the vertical axis denotes the appearance frequency of point mutation, and the solid line denotes the average value of the frequency of mutations in each group. An ROC curve for identifying cancer patients was created based on the frequency of variations of Group 2 (normal esophageal mucosa obtained from a healthy subject exposed to a risk factor for esophageal carcinogenesis) and the frequency of variations of Group 3 (noncancerous esophageal mucosa obtained from a patient with esophagus squamous epithelium carcinoma), and the AUC was calculated. The resulting ROC curve is shown in FIG. 3B. The AUC of this ROC curve was 0.790, and the linear trend p value was less than 0.001. As shown in FIG. 3B, it was shown that the appearance frequency of variations becomes high according to the risk of carcinogenesis.

What is claimed is:

1. A method for detecting a rare mutation, the method comprising the steps of:
preparing a sample comprising not more than 1,000 copies of template DNA;
amplifying the template DNA to prepare a library, and analyzing a nucleotide sequence of the library;
calculating a ratio of variants in a base at a predetermined position, from the analysis result;
comparing the calculated ratio of variants with a predetermined cut-off value; and
determining that the sample has a rare mutation in the base at the predetermined position when the calculated ratio of variants is not less than the predetermined cut-off value.

2. The detection method according to claim 1, wherein the rare mutation is variation recognized at a frequency of 1×10⁻³/base or less.

3. The detection method according to claim 1, wherein the ratio of variants in the base at the predetermined position is calculated by the following expression:
(Ratio of variants in base at predetermined position)=(Number of reads having variation in base at predetermined position)/(Number of reads containing base at predetermined position).

4. The detection method according to claim 1, wherein the predetermined cut-off value is a ratio of variants when an expected value of the number of variations due to an error in a sequencing length is 1 or less, and
the ratio of variants when the expected value is 1 or less is calculated from a Poisson probability obtained from an average value of Phred scores of analyzed nucleotide sequence and a Poisson distribution based on an average number of reads, and the sequencing length.

5. The detection method according to claim 4, wherein the average of the Poisson distribution is calculated by the following expression:
(Average of Poisson distribution)=(Average number of reads)×10^{-a/10}
wherein a is the average value of the Phred scores, and
the number of events of the Poisson distribution is the number of reads having variation due to an error in nucleic acid amplification and sequencing.

6. The detection method according to claim 4, wherein the expected value is calculated by the following expression:
(Expected value of number of variations due to error)=(Sequencing length)×(Poisson probability).

7. The detection method according to claim 1, wherein in the DNA template preparation step, the copy number of the DNA template is measured by real-time PCR or a spectrophotometer.

8. The detection method according to claim 7, wherein in the DNA template preparation step, when the copy number of the DNA template is more than 1,000, the sample is prepared to comprise not more than 1,000 copies of the DNA template by diluting the DNA template.

9. The detection method according to claim 1, wherein in the amplification step, the template DNA is amplified by PCR.

10. The detection method according to claim 1, wherein in the determination step, it is determined that the sample does not have a rare mutation in the base at the predetermined position when the ratio of variants is less than the predetermined cut-off value.

11. A method for detecting a rare mutation, the method comprising the steps of:
dividing a sample comprising template DNA to prepare a plurality of aliquots each comprising not more than 1,000 copies of template DNA;
amplifying the template DNA in a first aliquot to prepare a library, and analyzing a nucleotide sequence of the library;
calculating a ratio of variants in a base at a predetermined position, from the analysis result;
comparing the calculated ratio of variants with a predetermined cut-off value;
executing the amplification and analysis step, the calculation step, and the comparison step using other aliquots; and
determining that the sample has a rare mutation in the base at the predetermined position when the calculated ratio of variants in at least one of the aliquots is not less than the predetermined cut-off value.

12. The detection method according to claim 11, wherein the rare mutation is variation recognized at a frequency of 1×10⁻³/base or less.

13. The detection method according to claim 11, wherein the ratio of variants in the base at the predetermined position is calculated by the following expression:
(Ratio of variants in base at predetermined position)=(Number of reads having variation in base at predetermined position)/(Number of reads containing base at predetermined position).

14. The detection method according to claim 11, wherein the predetermined cut-off value is a ratio of variants when an expected value of the number of variations due to an error in a sequencing length is 1 or less, and
the ratio of variants when the expected value is 1 or less is calculated from a Poisson probability obtained from an average value of Phred scores of analyzed nucleotide sequence and a Poisson distribution based on an average number of reads, and the sequencing length.

15. The detection method according to claim 14, wherein the average of the Poisson distribution is calculated by the following expression:
(Average of Poisson distribution)=(Average number of reads)×10^{-a/10}
wherein a is the average value of the Phred scores, and
the number of events of the Poisson distribution is the number of reads having variation due to an error in nucleic acid amplification and sequencing.

16. The detection method according to claim 14, wherein the expected value is calculated by the following expression:
(Expected value of number of variations due to error)=(Sequencing length)×(Poisson probability).

17. The detection method according to claim 11, wherein in the DNA template preparation step, the copy number of the DNA template is measured by real-time PCR or a spectrophotometer.

18. The detection method according to claim 11, wherein in the amplification step, the template DNA is amplified by PCR.

19. The detection method according to claim 11, wherein in the analysis step, the nucleotide sequence of the library is determined by a DNA sequencer.

20. A method for detecting a rare mutation, the method comprising the steps of:
dividing a sample comprising template DNA to prepare a plurality of aliquots each comprising not more than 1,000 copies of template DNA;
amplifying the template DNA in a first aliquot to prepare a library, and analyzing a nucleotide sequence of the library;
calculating a ratio of variants in a base at a predetermined position, from the analysis result;
comparing the calculated ratio of variants with a predetermined cut-off value, determining that the sample has a rare mutation in the base at the predetermined position when the calculated ratio of variants in the first aliquot is not less than the predetermined cut-off value;
executing the amplification and analysis step, the calculation step, the comparison step and the determination step using a second aliquot when the calculated ratio of variants in the first aliquot is less than the predetermined cut-off value; and
determining that the sample has a rare mutation in the base at the predetermined position when the calculated ratio of variants in the second aliquot is not less than the predetermined cut-off value.


SEQUENCE LISTING (588 sequences)

SEQ ID NO: 1 - DNA, 20 bp, Artificial Sequence
  synthetic oligonucleotide primer
atccgaggctcccatgtaac

SEQ ID NO: 2 - DNA, 20 bp, Artificial Sequence
  synthetic oligonucleotide primer
gccaaacccactcaccgtca

SEQ ID NO: 3 - DNA, 20 bp, Artificial Sequence
  synthetic oligonucleotide primer
aatgtctcgtaatgccaatc

SEQ ID NO: 4 - DNA, 20 bp, Artificial Sequence
  synthetic oligonucleotide primer
cctaggcacaccaaatagtt

SEQ ID NO: 5 - DNA, 21 bp, Artificial Sequence
  synthetic oligonucleotide primer
tcttcgtgaaacctatggtga

SEQ ID NO: 6 - DNA, 19 bp, Artificial Sequence
  synthetic oligonucleotide primer
tcatgaaaagcagtgcaca

SEQ ID NO: 7 - DNA, 19 bp, Artificial Sequence
  synthetic oligonucleotide primer
ggccaactcaccagctgtt

SEQ ID NO: 8 - DNA, 24 bp, Artificial Sequence
  synthetic oligonucleotide primer
ctaagtgcagggacagatacatgg

SEQ ID NO: 9 - DNA, 21 bp, Artificial Sequence
  synthetic oligonucleotide primer
gaggtacgaactccgctatgg

SEQ ID NO: 10 - DNA, 21 bp, Artificial Sequence
  synthetic oligonucleotide primer
gggcagaagaaggtcagcata

SEQ ID NO: 11 - DNA, 20 bp, Artificial Sequence
  synthetic oligonucleotide primer
gacttaagctgctccctgct

SEQ ID NO: 12 - DNA, 19 bp, Artificial Sequence
  synthetic oligonucleotide primer
gggatcccctgcgtagtga

SEQ ID NO: 13 - DNA, 17 bp, Artificial Sequence
  synthetic oligonucleotide primer
gggtgggccgaagtctg

SEQ ID NO: 14 - DNA, 23 bp, Artificial Sequence
  synthetic oligonucleotide primer
agcgaaccaagaatgcctgttta

SEQ ID NO: 15 - DNA, 20 bp, Artificial Sequence
  synthetic oligonucleotide primer
gactcctttgcccctgtgtt

SEQ ID NO: 16 - DNA, 23 bp, Artificial Sequence
  synthetic oligonucleotide primer
gtttagctctgtccagggaactg

SEQ ID NO: 17 - DNA, 22 bp, Artificial Sequence
  synthetic oligonucleotide primer
gccaagaaaccatatgctcacc

SEQ ID NO: 18 - DNA, 23 bp, Artificial Sequence
  synthetic oligonucleotide primer
tttggattgtgtccgttgagcta

SEQ ID NO: 19 - DNA, 24 bp, Artificial Sequence
  synthetic oligonucleotide primer
gcaaactcttgcacaaatgctgaa

SEQ ID NO: 20 - DNA, 25 bp, Artificial Sequence
  synthetic oligonucleotide primer
tcccgtttttagggagcagattaag

SEQ ID NO: 21 - DNA, 18 bp, Artificial Sequence
  synthetic oligonucleotide primer
gaggaagccttcgcctgt

SEQ ID NO: 22 - DNA, 20 bp, Artificial Sequence
  synthetic oligonucleotide primer
gcattgcattccctgtggtt

SEQ ID NO: 23 - DNA, 25 bp, Artificial Sequence
  synthetic oligonucleotide primer
taaagatgatccgacaagtgagaga

SEQ ID NO: 24 - DNA, 20 bp, Artificial Sequence
  synthetic oligonucleotide primer
ggctcgccaattaaccctga

SEQ ID NO: 25 - DNA, 17 bp, Artificial Sequence
  synthetic oligonucleotide primer
cgcgtgctgttgggagt

SEQ ID NO: 26 - DNA, 28 bp, Artificial Sequence
  synthetic oligonucleotide primer
tctatcgcctcagttcctgttactaatt

SEQ ID NO: 27 - DNA, 28 bp, Artificial Sequence
  synthetic oligonucleotide primer
ctggtactaacataaattccccacttcc

SEQ ID NO: 28 - DNA, 28 bp, Artificial Sequence
  synthetic oligonucleotide primer
tctctcagtgtagcagttctatatggtt

SEQ ID NO: 29 - DNA, 23 bp, Artificial Sequence
  synthetic oligonucleotide primer
gggaggtggtagtggaatacact

SEQ ID NO: 30 - DNA, 26 bp, Artificial Sequence
  synthetic oligonucleotide primer
gatgttaggaagtaaggacagctgtg

SEQ ID NO: 31 - DNA, 18 bp, Artificial Sequence
  synthetic oligonucleotide primer
aggaggctgagtgggcta

SEQ ID NO: 32 - DNA, 22 bp, Artificial Sequence
  synthetic oligonucleotide primer
gatgtgctgttgagacctctgt

SEQ ID NO: 33 - DNA, 18 bp, Artificial Sequence
  synthetic oligonucleotide primer
ctggagagccatgaggca

SEQ ID NO: 34 - DNA, 19 bp, Artificial Sequence
  synthetic oligonucleotide primer
gaggagatgggtggcttgt

SEQ ID NO: 35 - DNA, 20 bp, Artificial Sequence
  synthetic oligonucleotide primer
caggagcgatcgtttgcaac

SEQ ID NO: 36 - DNA, 21 bp, Artificial Sequence
  synthetic oligonucleotide primer
gggagaacagggctgtatgga

SEQ ID NO: 37 - DNA, 18 bp, Artificial Sequence
  synthetic oligonucleotide primer
gcctgacgactcgtgcta

SEQ ID NO: 38 - DNA, 19 bp, Artificial Sequence
  synthetic oligonucleotide primer
cccatggtgcacctgggat

SEQ ID NO: 39 - DNA, 23 bp, Artificial Sequence
  synthetic oligonucleotide primer
cttctcctttacccctccttcct

SEQ ID NO: 40 - DNA, 22 bp, Artificial Sequence
  synthetic oligonucleotide primer
cgtggccccactacatgtataa

SEQ ID NO: 41 - DNA, 21 bp, Artificial Sequence
  synthetic oligonucleotide primer
gcagcttctgccatctctctc

SEQ ID NO: 42 - DNA, 24 bp, Artificial Sequence
  synthetic oligonucleotide primer
gtcacccaaactacggacattttc

SEQ ID NO: 43 - DNA, 24 bp, Artificial Sequence
  synthetic oligonucleotide primer
ttgctatgggatttcctgcagaaa

SEQ ID NO: 44 - DNA, 24 bp, Artificial Sequence
  synthetic oligonucleotide primer
ccattaggtacggtaagccaaaaa

SEQ ID NO: 45 - DNA, 28 bp, Artificial Sequence
  synthetic oligonucleotide primer
agctcatttttgttaatggtggcttttt

SEQ ID NO: 46 - DNA, 29 bp, Artificial Sequence
  synthetic oligonucleotide primer
tctttaactctacctcactctaacaagca

SEQ ID NO: 47 - DNA, 26 bp, Artificial Sequence
  synthetic oligonucleotide primer
tgaagatcttgaccaatggctaagtg

SEQ ID NO: 48 - DNA, 23 bp, Artificial Sequence
  synthetic oligonucleotide primer
tctcagatccaggaagaggaaag

SEQ ID NO: 49 - DNA, 22 bp, Artificial Sequence
  synthetic oligonucleotide primer
ctacgacccagttaccatagca

SEQ ID NO: 50 - DNA, 21 bp, Artificial Sequence
  synthetic oligonucleotide primer
tccgccactgaacattggaat

SEQ ID NO: 51 - DNA, 25 bp, Artificial Sequence
  synthetic oligonucleotide primer
ttaaccatgcagatcctcagtttgt

SEQ ID NO: 52 - DNA, 28 bp, Artificial Sequence
  synthetic oligonucleotide primer
ctgtccttattttggatatttctcccaa

SEQ ID NO: 53 - DNA, 27 bp, Artificial Sequence
  synthetic oligonucleotide primer
acctcagaaaaagtagaaaatggaagt

SEQ ID NO: 54 - DNA, 27 bp, Artificial Sequence
  synthetic oligonucleotide primer
catcacatacatacaagtcaacaaccc

SEQ ID NO: 55 - DNA, 26 bp, Artificial Sequence
  synthetic oligonucleotide primer
agatgagtcatatttgtgggttttca

SEQ ID NO: 56 - DNA, 28 bp, Artificial Sequence
  synthetic oligonucleotide primer
gctgatcttcatcaaaaggttcattctc

SEQ ID NO: 57 - DNA, 19 bp, Artificial Sequence
  synthetic oligonucleotide primer
ccctgcccactgtgttact

SEQ ID NO: 58 - DNA, 29 bp, Artificial Sequence
  synthetic oligonucleotide primer
gttctggcggtgttttgaaattagttatt

SEQ ID NO: 59 - DNA, 22 bp, Artificial Sequence
  synthetic oligonucleotide primer
aactgcagagtatttgggcgaa

SEQ ID NO: 60 - DNA, 26 bp, Artificial Sequence
  synthetic oligonucleotide primer
cccatgagttagaggaaatgaactga

SEQ ID NO: 61 - DNA, 22 bp, Artificial Sequence
  synthetic oligonucleotide primer
gggatacgtttggtcagcttgt

SEQ ID NO: 62 - DNA, 23 bp, Artificial Sequence
  synthetic oligonucleotide primer
cctgcttatctgttcctcctcct

SEQ ID NO: 63 - DNA, 19 bp, Artificial Sequence
  synthetic oligonucleotide primer
ccgtcgggcccgtatttac

SEQ ID NO: 64 - DNA, 22 bp, Artificial Sequence
  synthetic oligonucleotide primer
tggtctctcattctcccatccc

SEQ ID NO: 65 - DNA, 22 bp, Artificial Sequence
  synthetic oligonucleotide primer
gtcaagcaagaatgggctggta

SEQ ID NO: 66 - DNA, 26 bp, Artificial Sequence
  synthetic oligonucleotide primer
tgctaggattgttaaataaccgcctt

SEQ ID NO: 67 - DNA, 17 bp, Artificial Sequence
  synthetic oligonucleotide primer
cctgggagtccccctca

SEQ ID NO: 68 - DNA, 17 bp, Artificial Sequence
  synthetic oligonucleotide primer
ggccggtccctcctgat

SEQ ID NO: 69 - DNA, 17 bp, Artificial Sequence
  synthetic oligonucleotide primer
ggtggagagctgcctca

SEQ ID NO: 70 - DNA, 19 bp, Artificial Sequence
  synthetic oligonucleotide primer
cgtagccagctctcgcttt

SEQ ID NO: 71 - DNA, 22 bp, Artificial Sequence
  synthetic oligonucleotide primer
gttcacctgtactggtggatgt

SEQ ID NO: 72 - DNA, 20 bp, Artificial Sequence
  synthetic oligonucleotide primer
caggattcctaccggaagca

SEQ ID NO: 73 - DNA, 17 bp, Artificial Sequence
  synthetic oligonucleotide primer
gctgctggcacctggac

SEQ ID NO: 74 - DNA, 17 bp, Artificial Sequence
  synthetic oligonucleotide primer
tgagcagggccctcctt

SEQ ID NO: 75 - DNA, 19 bp, Artificial Sequence
  synthetic oligonucleotide primer
gtgctgcgaagtggaaacc

SEQ ID NO: 76 - DNA, 25 bp, Artificial Sequence
  synthetic oligonucleotide primer
caagttgcagggaagtcttaagaga

SEQ ID NO: 77 - DNA, 17 bp, Artificial Sequence
  synthetic oligonucleotide primer
cgtgcctccgtaggtct

SEQ ID NO: 78 - DNA, 21 bp, Artificial Sequence
  synthetic oligonucleotide primer
cggtgtagatgcacagcttct

SEQ ID NO: 79 - DNA, 23 bp, Artificial Sequence
  synthetic oligonucleotide primer
tctccttctgcctcagatgtgaa

SEQ ID NO: 80 - DNA, 22 bp, Artificial Sequence
  synthetic oligonucleotide primer
cactaggtgtctccccctgtaa

SEQ ID NO: 81 - DNA, 20 bp, Artificial Sequence
  synthetic oligonucleotide primer
cccttctaaggaccccctct

SEQ ID NO: 82 - DNA, 17 bp, Artificial Sequence
  synthetic oligonucleotide primer
tggcgccctcagatgtc

SEQ ID NO: 83 - DNA, 28 bp, Artificial Sequence
  synthetic oligonucleotide primer
ggtgcttatgaatcaacaaaatggagaa

SEQ ID NO: 84 - DNA, 29 bp, Artificial Sequence
  synthetic oligonucleotide primer
acaggaaatttctaaatgtgacatgacct

SEQ ID NO: 85 - DNA, 27 bp, Artificial Sequence
  synthetic oligonucleotide primer
tgacaagatggactttttaaccattgt

SEQ ID NO: 86 - DNA, 28 bp, Artificial Sequence
  synthetic oligonucleotide primer
ctccttcctaacagtttaccaaagttga

SEQ ID NO: 87 - DNA, 21 bp, Artificial Sequence
  synthetic oligonucleotide primer
gctcctgcaagaagccatctt

SEQ ID NO: 88 - DNA, 23 bp, Artificial Sequence
  synthetic oligonucleotide primer
cctatggtactttggctctctcc

SEQ ID NO: 89 - DNA, 26 bp, Artificial Sequence
  synthetic oligonucleotide primer
aagtcattttgatgaggtgaagtcca

SEQ ID NO: 90 - DNA, 26 bp, Artificial Sequence
  synthetic oligonucleotide primer
ttgaagccatacctgttttcccaata

SEQ ID NO: 91 - DNA, 26 bp, Artificial Sequence
  synthetic oligonucleotide primer
ctatatgtagaggctgttggaagctg

SEQ ID NO: 92 - DNA, 28 bp, Artificial Sequence
  synthetic oligonucleotide primer
ctcaccaatcttctaccagtgtgttatt

SEQ ID NO: 93 - DNA, 29 bp, Artificial Sequence
  synthetic oligonucleotide primer
ttcagtggaggttaacattcatcaagatt

SEQ ID NO: 94 - DNA, 22 bp, Artificial Sequence
  synthetic oligonucleotide primer
ctgtagataggccagcattgga

SEQ ID NO: 95 - DNA, 27 bp, Artificial Sequence
  synthetic oligonucleotide primer
tttctgttaagcagtcactaccattgt

SEQ ID NO: 96 - DNA, 23 bp, Artificial Sequence
  synthetic oligonucleotide primer
gctgtaaagtgagcagcacaaga

SEQ ID NO: 97 - DNA, 28 bp, Artificial Sequence
  synthetic oligonucleotide primer
ttaaattggttgtgttttcttgaaggca

SEQ ID NO: 98 - DNA, 26 bp, Artificial Sequence
  synthetic oligonucleotide primer
cctacttcctctttggctcttttcag

SEQ ID NO: 99 - DNA, 28 bp, Artificial Sequence
  synthetic oligonucleotide primer
agttctgttaaagttcatggcttttgtg

SEQ ID NO: 100 - DNA, 23 bp, Artificial Sequence
  synthetic oligonucleotide primer
ccagagggaacaaagtcggaata

SEQ ID NO: 101 - DNA, 27 bp, Artificial Sequence
  synthetic oligonucleotide primer
gagatggaatcagtgatttcagattgt

SEQ ID NO: 102 - DNA, 28 bp, Artificial Sequence
  synthetic oligonucleotide primer
gcaaacaacattccatgatgaccaaata

SEQ ID NO: 103 - DNA, 29 bp, Artificial Sequence
  synthetic oligonucleotide primer
attaccacttgtactagtatgccttaaga

SEQ ID NO: 104 - DNA, 26 bp, Artificial Sequence
  synthetic oligonucleotide primer
cctgtacacatgaagccatcgtatat

SEQ ID NO: 105 - DNA, 24 bp, Artificial Sequence
  synthetic oligonucleotide primer
gccctctcaagagacaaaaacatt

SEQ ID NO: 106 - DNA, 24 bp, Artificial Sequence
  synthetic oligonucleotide primer
aacagtagacacaaaacaggctca

SEQ ID NO: 107 - DNA, 21 bp, Artificial Sequence
  synthetic oligonucleotide primer
cctccccagtcctcatgtact

SEQ ID NO: 108 - DNA, 27 bp, Artificial Sequence
  synthetic oligonucleotide primer
taaaaggtgcactgtaataatccagac

SEQ ID NO: 109 - DNA, 27 bp, Artificial Sequence
  synthetic oligonucleotide primer
agtactcatgaaaatggtcagagaaac

SEQ ID NO: 110 - DNA, 22 bp, Artificial Sequence
  synthetic oligonucleotide primer
aaggcctgctgaaaatgactga

SEQ ID NO: 111 - DNA, 27 bp, Artificial Sequence
  synthetic oligonucleotide primer
ctggtgtaactctttatttgtcccctt

SEQ ID NO: 112 - DNA, 26 bp, Artificial Sequence
  synthetic oligonucleotide primer
gctcaatgacatctccattcttctct

SEQ ID NO: 113 - DNA, 21 bp, Artificial Sequence
  synthetic oligonucleotide primer
cttcatcctggctctgcagtt

SEQ ID NO: 114 - DNA, 18 bp, Artificial Sequence
  synthetic oligonucleotide primer
gccctcaggctggtacct

SEQ ID NO: 115 - DNA, 17 bp, Artificial Sequence
  synthetic oligonucleotide primer
gcagccgagccatggtt

SEQ ID NO: 116 - DNA, 17 bp, Artificial Sequence
  synthetic oligonucleotide primer
agcccattgggcagctc

SEQ ID NO: 117 - DNA, 20 bp, Artificial Sequence
  synthetic oligonucleotide primer
gtcgataccactggcctcaa

SEQ ID NO: 118 - DNA, 19 bp, Artificial Sequence
  synthetic oligonucleotide primer
gggatggtgaagcttccag

SEQ ID NO: 119 - DNA, 19 bp, Artificial Sequence
  synthetic oligonucleotide primer
gcagggagggctgattgaa

SEQ ID NO: 120 - DNA, 21 bp, Artificial Sequence
  synthetic oligonucleotide primer
gaccaaaccagcactgtttcc

SEQ ID NO: 121 - DNA, 20 bp, Artificial Sequence
  synthetic oligonucleotide primer
gaggctcatgggtggctatt

SEQ ID NO: 122 - DNA, 17 bp, Artificial Sequence
  synthetic oligonucleotide primer
ggcccgctgtacgtgtc

SEQ ID NO: 123 - DNA, 24 bp, Artificial Sequence
  synthetic oligonucleotide primer
cgacacaacacaaaatagccgtat

SEQ ID NO: 124 - DNA, 28 bp, Artificial Sequence
  synthetic oligonucleotide primer
catcacagtaaataacactctggtgtca

SEQ ID NO: 125 - DNA, 26 bp, Artificial Sequence
  synthetic oligonucleotide primer
agttcacactgtgactgagaaaagac

SEQ ID NO: 126 - DNA, 21 bp, Artificial Sequence
  synthetic oligonucleotide primer
gctctgaaagagaggcactca

SEQ ID NO: 127 - DNA, 26 bp, Artificial Sequence
  synthetic oligonucleotide primer
aatggaaaagaaatgctgcagaaaca

SEQ ID NO: 128 - DNA, 24 bp, Artificial Sequence
  synthetic oligonucleotide primer
gcagaactgcctattcctaactga

SEQ ID NO: 129 - DNA, 28 bp, Artificial Sequence
  synthetic oligonucleotide primer
tcatgaaagagtcaataggtcagagagt

SEQ ID NO: 130 - DNA, 23 bp, Artificial Sequence
  synthetic oligonucleotide primer
ccagccagtgagcttatttcaca

SEQ ID NO: 131 - DNA, 28 bp, Artificial Sequence
  synthetic oligonucleotide primer
catttggtaggcttgagtttgaagaaac

SEQ ID NO: 132 - DNA, 28 bp, Artificial Sequence
  synthetic oligonucleotide primer
gaaaatccttaccaatactccatccaca

SEQ ID NO: 133 - DNA, 33 bp, Artificial Sequence
  synthetic oligonucleotide primer
aacgaaataacacaaatttttaaggttactgat

SEQ ID NO: 134 - DNA, 28 bp, Artificial Sequence
  synthetic oligonucleotide primer
actttacctttccaatttgctgaagagt

SEQ ID NO: 135 - DNA, 29 bp, Artificial Sequence
  synthetic oligonucleotide primer
actttctttcagtgatacatttttcctgt

SEQ ID NO: 136 - DNA, 26 bp, Artificial Sequence
  synthetic oligonucleotide primer
ggaatttagtccaaaggaatgccaat

SEQ ID NO: 137 - DNA, 25 bp, Artificial Sequence
  synthetic oligonucleotide primer
ctgtgtgctgagagatgtaatgaca

SEQ ID NO: 138 - DNA, 33 bp, Artificial Sequence
  synthetic oligonucleotide primer
tcagtatcaacctatatctaaagcaaatcaatc

SEQ ID NO: 139 - DNA, 27 bp, Artificial Sequence
  synthetic oligonucleotide primer
aacagatttgtctttcccatggattct

SEQ ID NO: 140 - DNA, 29 bp, Artificial Sequence
  synthetic oligonucleotide primer
gttagccatatgcacatgaatgaatttct

SEQ ID NO: 141 - DNA, 26 bp, Artificial Sequence
  synthetic oligonucleotide primer
ctgacttttaaattgccactgtcaat

SEQ ID NO: 142 - DNA, 28 bp, Artificial Sequence
  synthetic oligonucleotide primer
gaggaagattaagaggacaagcagattc

SEQ ID NO: 143 - DNA, 25 bp, Artificial Sequence
  synthetic oligonucleotide primer
tcttattcccacagtgtatcggcta

SEQ ID NO: 144 - DNA, 22 bp, Artificial Sequence
  synthetic oligonucleotide primer
gaggagagaaggtgaagtgctt

SEQ ID NO: 145 - DNA, 33 bp, Artificial Sequence
  synthetic oligonucleotide primer
agaacaaaaccatgtaataaaattctgactact

SEQ ID NO: 146 - DNA, 22 bp, Artificial Sequence
  synthetic oligonucleotide primer
acctacctcctgaacagcatga

SEQ ID NO: 147 - DNA, 25 bp, Artificial Sequence
  synthetic oligonucleotide primer
tcttcctcagacattcaaacgtgtt

SEQ ID NO: 148 - DNA, 24 bp, Artificial Sequence
  synthetic oligonucleotide primer
atgttttggtggacccattacatt

SEQ ID NO: 149 - DNA, 25 bp, Artificial Sequence
  synthetic oligonucleotide primer
acagtcattgctcagatccaaaaga

SEQ ID NO: 150 - DNA, 19 bp, Artificial Sequence
  synthetic oligonucleotide primer
caggtcctagctgtgggtt

SEQ ID NO: 151 - DNA, 22 bp, Artificial Sequence
  synthetic oligonucleotide primer
ggtgggacaagaagtcaatgct

SEQ ID NO: 152 - DNA, 17 bp, Artificial Sequence
  synthetic oligonucleotide primer
gccatcgacgtgaggga

SEQ ID NO: 153 - DNA, 18 bp, Artificial Sequence
  synthetic oligonucleotide primer
gggagctgaagtcgaggt

SEQ ID NO: 154 - DNA, 17 bp, Artificial Sequence
  synthetic oligonucleotide primer
gccccggcgagtacatc

SEQ ID NO: 155 - DNA, 18 bp, Artificial Sequence
  synthetic oligonucleotide primer
ctgctggagctcctgtgg

SEQ ID NO: 156 - DNA, 19 bp, Artificial Sequence
  synthetic oligonucleotide primer
ctgcgcaagaggacctact

SEQ ID NO: 157 - DNA, 20 bp, Artificial Sequence
  synthetic oligonucleotide primer
cggaactcgaagagctcctt

SEQ ID NO: 158 - DNA, 20 bp, Artificial Sequence
  synthetic oligonucleotide primer
cggcttcgtgaagctcaact

SEQ ID NO: 159 - DNA, 17 bp, Artificial Sequence
  synthetic oligonucleotide primer
accccgcaccctcatct

SEQ ID NO: 160 - DNA, 17 bp, Artificial Sequence
  synthetic oligonucleotide primer
gcttgctgaccctggtg

SEQ ID NO: 161 - DNA, 21 bp, Artificial Sequence
  synthetic oligonucleotide primer
ccccaaatctgaatcccgaga

SEQ ID NO: 162 - DNA, 20 bp, Artificial Sequence
  synthetic oligonucleotide primer
gggtctgacgggtagagtgt

SEQ ID NO: 163 - DNA, 18 bp, Artificial Sequence
  synthetic oligonucleotide primer
cgtaccctgggccaggat

SEQ ID NO: 164 - DNA, 19 bp, Artificial Sequence
  synthetic oligonucleotide primer
gtcagccttctgccctctc

SEQ ID NO: 165 - DNA, 20 bp, Artificial Sequence
  synthetic oligonucleotide primer
aggtcagtggatcccctctc

SEQ ID NO: 166 - DNA, 26 bp, Artificial Sequence
  synthetic oligonucleotide primer
ggaccactattatctctgtcctcaca

SEQ ID NO: 167 - DNA, 19 bp, Artificial Sequence
  synthetic oligonucleotide primer
agggacctgcagtccagaa

SEQ ID NO: 168 - DNA, 18 bp, Artificial Sequence
  synthetic oligonucleotide primer
gcatgatgcgctgtgtgt

SEQ ID NO: 169 - DNA, 17 bp, Artificial Sequence
  synthetic oligonucleotide primer
ggctgctcttgcgaggt

SEQ ID NO: 170 - DNA, 19 bp, Artificial Sequence
  synthetic oligonucleotide primer
ctcgttcgctctccagctt

SEQ ID NO: 171 - DNA, 19 bp, Artificial Sequence
  synthetic oligonucleotide primer
tccctcgacacccgattca

SEQ ID NO: 172 - DNA, 23 bp, Artificial Sequence
  synthetic oligonucleotide primer
cgcactaaaacaacagcgaactt

SEQ ID NO: 173 - DNA, 25 bp, Artificial Sequence
  synthetic oligonucleotide primer
cctcacttggttctttcagctcttc

SEQ ID NO: 174 - DNA, 23 bp, Artificial Sequence
  synthetic oligonucleotide primer
gggtccaaagaacctaagagtct

SEQ ID NO: 175 - DNA, 20 bp, Artificial Sequence
  synthetic oligonucleotide primer
agtcccaaagtgcagcttgt

SEQ ID NO: 176 - DNA, 17 bp, Artificial Sequence
  synthetic oligonucleotide primer
tctcggtccagcccagt

SEQ ID NO: 177 - DNA, 21 bp, Artificial Sequence
  synthetic oligonucleotide primer
ccaaaggtggctagtgttcct

SEQ ID NO: 178 - DNA, 27 bp, Artificial Sequence
  synthetic oligonucleotide primer
tttggaaaccctctaaggagttataga

SEQ ID NO: 179 - DNA, 27 bp, Artificial Sequence
  synthetic oligonucleotide primer
gcagtcttggtactttgtaaatgacac

SEQ ID NO: 180 - DNA, 24 bp, Artificial Sequence
  synthetic oligonucleotide primer
tgctgttttcaaaatgccatcgtt

SEQ ID NO: 181 - DNA, 18 bp, Artificial Sequence
  synthetic oligonucleotide primer
ggtgggaggctgtcagtg

SEQ ID NO: 182 - DNA, 24 bp, Artificial Sequence
  synthetic oligonucleotide primer
cctctcactcatgtgatgtcatct

SEQ ID NO: 183 - DNA, 25 bp, Artificial Sequence
  synthetic oligonucleotide primer
catgaaggcaggatgagaatggaat

SEQ ID NO: 184 - DNA, 22 bp, Artificial Sequence
  synthetic oligonucleotide primer
cttacttctccccctcctctgt

SEQ ID NO: 185 - DNA, 24 bp, Artificial Sequence
  synthetic oligonucleotide primer
tgcaggtaaaacagtcaagaagaa

SEQ ID NO: 186 - DNA, 22 bp, Artificial Sequence
  synthetic oligonucleotide primer
ggagaccaagggtgcagttatg

SEQ ID NO: 187 - DNA, 20 bp, Artificial Sequence
  synthetic oligonucleotide primer
ctcctccaccgcttcttgtc

SEQ ID NO: 188 - DNA, 25 bp, Artificial Sequence
  synthetic oligonucleotide primer
gatttccttactgcctcttgcttct

SEQ ID NO: 189 - DNA, 17 bp, Artificial Sequence
  synthetic oligonucleotide primer
gtgcagggtggcaagtg

SEQ ID NO: 190 - DNA, 18 bp, Artificial Sequence
  synthetic oligonucleotide primer
ccacaggtctccccaagg

SEQ ID NO: 191 - DNA, 20 bp, Artificial Sequence
  synthetic oligonucleotide primer
accacccttaacccctcctc

SEQ ID NO: 192 - DNA, 18 bp, Artificial Sequence
  synthetic oligonucleotide primer
gagacgacagggctggtt

SEQ ID NO: 193 - DNA, 20 bp, Artificial Sequence
  synthetic oligonucleotide primer
cgcctcacaacctccgtcat

SEQ ID NO: 194 - DNA, 22 bp, Artificial Sequence
  synthetic oligonucleotide primer
tgttcacttgtgccctgacttt

SEQ ID NO: 195 - DNA, 18 bp, Artificial Sequence
  synthetic oligonucleotide primer
ctcagggcaactgaccgt

SEQ ID NO: 196 - DNA, 22 bp, Artificial Sequence
  synthetic oligonucleotide primer
gaagacccaggtccagatgaag

SEQ ID NO: 197 - DNA, 21 bp, Artificial Sequence
  synthetic oligonucleotide primer
gcttcccacaggtctctgcta

SEQ ID NO: 198 - DNA, 21 bp, Artificial Sequence
  synthetic oligonucleotide primer
gggttggaagtgtctcatgct

SEQ ID NO: 199 - DNA, 20 bp, Artificial Sequence
  synthetic oligonucleotide primer
ggcacggtaatgctgctcat

SEQ ID NO: 200 - DNA, 19 bp, Artificial Sequence
  synthetic oligonucleotide primer
ggcagtgagtgggtacctc

SEQ ID NO: 201 - DNA, 24 bp, Artificial Sequence
  synthetic oligonucleotide primer
aggacaagtaatgatctcctggaa

SEQ ID NO: 202 - DNA, 21 bp, Artificial Sequence
  synthetic oligonucleotide primer
tccttcctgtcctcctagcag

SEQ ID NO: 203 - DNA, 20 bp, Artificial Sequence
  synthetic oligonucleotide primer
gggtgtgtggtctcccatac

SEQ ID NO: 204 - DNA, 23 bp, Artificial Sequence
  synthetic oligonucleotide primer
aatctgcatacaccagttcagca

SEQ ID NO: 205 - DNA, 21 bp, Artificial Sequence
  synthetic oligonucleotide primer
gccctcccagaaggtctacat

SEQ ID NO: 206 - DNA, 19 bp, Artificial Sequence
  synthetic oligonucleotide primer
cctcctctgctccttggtc

SEQ ID NO: 207 - DNA, 20 bp, Artificial Sequence
  synthetic oligonucleotide primer
agcccatgggagaactctga

SEQ ID NO: 208 - DNA, 19 bp, Artificial Sequence
  synthetic oligonucleotide primer
cccatcccagctctcatcc

SEQ ID NO: 209 - DNA, 26 bp, Artificial Sequence
  synthetic oligonucleotide primer
aagtcttttcatgggacttgattggt

SEQ ID NO: 210 - DNA, 27 bp, Artificial Sequence
  synthetic oligonucleotide primer
cctgcctgtggacttgaatttcataat

SEQ ID NO: 211 - DNA, 27 bp, Artificial Sequence
  synthetic oligonucleotide primer
tgaactccagaatatgcaagaatgcaa

SEQ ID NO: 212 - DNA, 26 bp, Artificial Sequence
  synthetic oligonucleotide primer
agatcttcaacaaccaggaatttgct

SEQ ID NO: 213 - DNA, 26 bp, Artificial Sequence
  synthetic oligonucleotide primer
attggagagtaaacctaagcagaacc

SEQ ID NO: 214 - DNA, 27 bp, Artificial Sequence
  synthetic oligonucleotide primer
tgaattgttcacgcatttcttcctttt

SEQ ID NO: 215 - DNA, 26 bp, Artificial Sequence
  synthetic oligonucleotide primer
aacatatgtgcaacttacccaagcta

SEQ ID NO: 216 - DNA, 32 bp, Artificial Sequence
  synthetic oligonucleotide primer
acttgatcagaagttctggaaatacttcattt

SEQ ID NO: 217 - DNA, 28 bp, Artificial Sequence
  synthetic oligonucleotide primer
ttggtatgcgtctcaacttctctaaatt

SEQ ID NO: 218 - DNA, 22 bp, Artificial Sequence
  synthetic oligonucleotide primer
gttgcagctgtgcttgatttgt

SEQ ID NO: 219 - DNA, 27 bp, Artificial Sequence
  synthetic oligonucleotide primer
cgaatacaccaacaagtaatgatgcct

SEQ ID NO: 220 - DNA, 28 bp, Artificial Sequence
  synthetic oligonucleotide primer
cacatttactaggatgagctccatttgt

SEQ ID NO: 221 - DNA, 20 bp, Artificial Sequence
  synthetic oligonucleotide primer
tggctggtcggaaaggattt

SEQ ID NO: 222 - DNA, 28 bp, Artificial Sequence
  synthetic oligonucleotide primer
gtttcttaggatgaaagcaaagtctact

SEQ ID NO: 223 - DNA, 27 bp, Artificial Sequence
  synthetic oligonucleotide primer
atgatggtgaaggatgaatatgtgcat

SEQ ID NO: 224 - DNA, 24 bp, Artificial Sequence
  synthetic oligonucleotide primer
agtgctggtagcattagactcaga

SEQ ID NO: 225 - DNA, 20 bp, Artificial Sequence
  synthetic oligonucleotide primer
ggcagccatagtgaaggact

SEQ ID NO: 226 - DNA, 28 bp, Artificial Sequence
  synthetic oligonucleotide primer
aggtggtagtgctgtctaaaaattaagg

SEQ ID NO: 227 - DNA, 24 bp, Artificial Sequence
  synthetic oligonucleotide primer
tgttgtcttttctttagggcctgt

SEQ ID NO: 228 - DNA, 27 bp, Artificial Sequence
  synthetic oligonucleotide primer
gcgtttcaatcaccactaaatcaatct

SEQ ID NO: 229 - DNA, 24 bp, Artificial Sequence
  synthetic oligonucleotide primer
tttctcatgggaggatgttctttc

SEQ ID NO: 230 - DNA, 23 bp, Artificial Sequence
  synthetic oligonucleotide primer
cttgctctctcaatggcttctgt

SEQ ID NO: 231 - DNA, 24 bp, Artificial Sequence
  synthetic oligonucleotide primer
ttcctaaggttgcacataggcaaa

SEQ ID NO: 232 - DNA, 25 bp, Artificial Sequence
  synthetic oligonucleotide primer
atgcacttgggtagatcttatgaac

SEQ ID NO: 233 - DNA, 23 bp, Artificial Sequence
  synthetic oligonucleotide primer
gtctttgatttgcgtcagtgtca

SEQ ID NO: 234 - DNA, 28 bp, Artificial Sequence
  synthetic oligonucleotide primer
ctgctcaaagaaactaatcaactgagta

SEQ ID NO: 235 - DNA, 20 bp, Artificial Sequence
  synthetic oligonucleotide primer
tgtcagctgctgctggaatt

SEQ ID NO: 236 - DNA, 24 bp, Artificial Sequence
  synthetic oligonucleotide primer
ctcagtctaaaggttgtgggtctg

SEQ ID NO: 237 - DNA, 19 bp, Artificial Sequence
  synthetic oligonucleotide primer
agcagctgggcatgttcac

SEQ ID NO: 238 - DNA, 18 bp, Artificial Sequence
  synthetic oligonucleotide primer
gatcttgacggccctcct

SEQ ID NO: 239 - DNA, 19 bp, Artificial Sequence
  synthetic oligonucleotide primer
tcctctgtcctgtgtgcct

SEQ ID NO: 240 - DNA, 18 bp, Artificial Sequence
  synthetic oligonucleotide primer
caggttccccggcttgat

SEQ ID NO: 241 - DNA, 17 bp, Artificial Sequence
  synthetic oligonucleotide primer
cgaggtaggcacgtgct

SEQ ID NO: 242 - DNA, 18 bp, Artificial Sequence
  synthetic oligonucleotide primer
cccagccgaccagatgtc

SEQ ID NO: 243 - DNA, 21 bp, Artificial Sequence
  synthetic oligonucleotide primer
cctttcttccctcccctcgaa

SEQ ID NO: 244 - DNA, 23 bp, Artificial Sequence
  synthetic oligonucleotide primer
ccctacatttctgcacaaaagcc

SEQ ID NO: 245 - DNA, 18 bp, Artificial Sequence
  synthetic oligonucleotide primer
ccactgcttctgggcgtt

SEQ ID NO: 246 - DNA, 25 bp, Artificial Sequence
  synthetic oligonucleotide primer
tcctgagtgtagatgatgtcatcct

SEQ ID NO: 247 - DNA, 21 bp, Artificial Sequence
  synthetic oligonucleotide primer
atctccccagactggatgtca

SEQ ID NO: 248 - DNA, 18 bp, Artificial Sequence
  synthetic oligonucleotide primer
cgacaggatcccctgggt

SEQ ID NO: 249 - DNA, 19 bp, Artificial Sequence
  synthetic oligonucleotide primer
actgtctccagccatgcac

SEQ ID NO: 250 - DNA, 19 bp, Artificial Sequence
  synthetic oligonucleotide primer
tggccaggtgttcccctaa

SEQ ID NO: 251 - DNA, 19 bp, Artificial Sequence
  synthetic oligonucleotide primer
tgccagtcctcatgttgcc

SEQ ID NO: 252 - DNA, 18 bp, Artificial Sequence
  synthetic oligonucleotide primer
tgaggctgggttgcactt

SEQ ID NO: 253 - DNA, 27 bp, Artificial Sequence
  synthetic oligonucleotide primer
tctgtttttgtcttgtttggtgtgttt

SEQ ID NO: 254 - DNA, 21 bp, Artificial Sequence
  synthetic oligonucleotide primer
caccagagtgtctccagcaag

SEQ ID NO: 255 - DNA, 28 bp, Artificial Sequence
  synthetic oligonucleotide primer
catcttatctcacctctcctgtgtattt

SEQ ID NO: 256 - DNA, 23 bp, Artificial Sequence
  synthetic oligonucleotide primer
gtaagagacctggaagccatgtg

SEQ ID NO: 257 - DNA, 27 bp, Artificial Sequence
  synthetic oligonucleotide primer
aagtctataaacttcacagggagacct

SEQ ID NO: 258 - DNA, 27 bp, Artificial Sequence
  synthetic oligonucleotide primer
gtggagctcgagaaataacacacatta

SEQ ID NO: 259 - DNA, 20 bp, Artificial Sequence
  synthetic oligonucleotide primer
agcccacgatgtcttcactg

SEQ ID NO: 260 - DNA, 25 bp, Artificial Sequence
  synthetic oligonucleotide primer
agaatttggccaagaaggactgaaa

SEQ ID NO: 261 - DNA, 19 bp, Artificial Sequence
  synthetic oligonucleotide primer
atccgtggaccttgtgcaa

SEQ ID NO: 262 - DNA, 22 bp, Artificial Sequence
  synthetic oligonucleotide primer
tcctctcctggtctctcaacag

SEQ ID NO: 263 - DNA, 20 bp, Artificial Sequence
  synthetic oligonucleotide primer
cagccacaccccattcttga

SEQ ID NO: 264 - DNA, 23 bp, Artificial Sequence
  synthetic oligonucleotide primer
gccgttgtacactcatcttccta

SEQ ID NO: 265 - DNA, 21 bp, Artificial Sequence
  synthetic oligonucleotide primer
acacagatcagcgacaggatg

SEQ ID NO: 266 - DNA, 24 bp, Artificial Sequence
  synthetic oligonucleotide primer
agatttccctcctctcactgacaa

SEQ ID NO: 267 - DNA, 21 bp, Artificial Sequence
  synthetic oligonucleotide primer
cctgtccttggcacaacaact

SEQ ID NO: 268 - DNA, 26 bp, Artificial Sequence
  synthetic oligonucleotide primer
ccagactcagctcagttaattttggt

SEQ ID NO: 269 - DNA, 25 bp, Artificial Sequence
  synthetic oligonucleotide primer
cgatctgttagaaacctctccaggt

SEQ ID NO: 270 - DNA, 20 bp, Artificial Sequence
  synthetic oligonucleotide primer
cttggcttgcggactctgta

SEQ ID NO: 271 - DNA, 27 bp, Artificial Sequence
  synthetic oligonucleotide primer
acctgtagacctagttaccaaaagaca

SEQ ID NO: 272 - DNA, 27 bp, Artificial Sequence
  synthetic oligonucleotide primer
cctgctaccatatcagagaccaactaa

SEQ ID NO: 273 - DNA, 21 bp, Artificial Sequence
  synthetic oligonucleotide primer
ggagagcactctctggtgaga

SEQ ID NO: 274 - DNA, 20 bp, Artificial Sequence
  synthetic oligonucleotide primer
agttggacccaacgcttcat

SEQ ID NO: 275 - DNA, 26 bp, Artificial Sequence
  synthetic oligonucleotide primer
acgcccatcatatttcttcagaatag

SEQ ID NO: 276 - DNA, 23 bp, Artificial Sequence
  synthetic oligonucleotide primer
ctctcactggcttctcctctaca

SEQ ID NO: 277 - DNA, 23 bp, Artificial Sequence
  synthetic oligonucleotide primer
agttggaaatttctgggccatga

SEQ ID NO: 278 - DNA, 28 bp, Artificial Sequence
  synthetic oligonucleotide primer
tcaagttgaaacaaatgtggaaatcacc

SEQ ID NO: 279 - DNA, 23 bp, Artificial Sequence
  synthetic oligonucleotide primer
cccagccagattatcctttctga

SEQ ID NO: 280 - DNA, 20 bp, Artificial Sequence
  synthetic oligonucleotide primer
gtggcggttctgtggtagag

SEQ ID NO: 281 - DNA, 26 bp, Artificial Sequence
  synthetic oligonucleotide primer
agtcttacatttgaccatgaccatgt

SEQ ID NO: 282 - DNA, 28 bp, Artificial Sequence
  synthetic oligonucleotide primer
tggtatagtgctggtttgttcaacatat

SEQ ID NO: 283 - DNA, 20 bp, Artificial Sequence
  synthetic oligonucleotide primer
cacataccaggtgagccctt

SEQ ID NO: 284 - DNA, 24 bp, Artificial Sequence
  synthetic oligonucleotide primer
ttttcacatttcagggtcctgaca

SEQ ID NO: 285 - DNA, 24 bp, Artificial Sequence
  synthetic oligonucleotide primer
actgacccatgaataccagtgact

SEQ ID NO: 286 - DNA, 26 bp, Artificial Sequence
  synthetic oligonucleotide primer
caatcccctaactctgagtcttgttt

SEQ ID NO: 287 - DNA, 28 bp, Artificial Sequence
  synthetic oligonucleotide primer
actaaataatctgagctaccactcacct

SEQ ID NO: 288 - DNA, 28 bp, Artificial Sequence
  synthetic oligonucleotide primer
tgttttgagcttgtttgctgaatgttaa

SEQ ID NO: 289 - DNA, 31 bp, Artificial Sequence
  synthetic oligonucleotide primer
ttattctgttacttacgtggacatttcttga

SEQ ID NO: 290 - DNA, 24 bp, Artificial Sequence
  synthetic oligonucleotide primer
gttactcagtgtccccaaaccttt

SEQ ID NO: 291 - DNA, 27 bp, Artificial Sequence
  synthetic oligonucleotide primer
ctgaatcaaatagggaaggaaaggaga

SEQ ID NO: 292 - DNA, 22 bp, Artificial Sequence
  synthetic oligonucleotide primer
cctggtggcagactttgatcat

SEQ ID NO: 293 - DNA, 23 bp, Artificial Sequence
  synthetic oligonucleotide primer
agcataactcattcatcgccaca

SEQ ID NO: 294 - DNA, 25 bp, Artificial Sequence
  synthetic oligonucleotide primer
tcctttgttatgcagacaccattca

SEQ ID NO: 295 - DNA, 26 bp, Artificial Sequence
  synthetic oligonucleotide primer
gccttagagtgttcctcaatgtaaca

SEQ ID NO: 296 - DNA, 26 bp, Artificial Sequence
  synthetic oligonucleotide primer
cgcattattcgtgggacaaaacttta

SEQ ID NO: 297 - DNA, 17 bp, Artificial Sequence
  synthetic oligonucleotide primer
gccccagagtgctctgt

SEQ ID NO: 298 - DNA, 17 bp, Artificial Sequence
  synthetic oligonucleotide primer
ccgcgccgtgtactcat

SEQ ID NO: 299 - DNA, 17 bp, Artificial Sequence
  synthetic oligonucleotide primer
tgaaccgcgaggtgctg

SEQ ID NO: 300 - DNA, 17 bp, Artificial Sequence
  synthetic oligonucleotide primer
cccgcctgtgcctagag

SEQ ID NO: 301 - DNA, 17 bp, Artificial Sequence
  synthetic oligonucleotide primer
ggtccgcatttcgcctt

SEQ ID NO: 302 - DNA, 19 bp, Artificial Sequence
  synthetic oligonucleotide primer
gctacctcggagccgatca

SEQ ID NO: 303 - DNA, 22 bp, Artificial Sequence
  synthetic oligonucleotide primer
ctgcgacccttataatgagcct

SEQ ID NO: 304 - DNA, 26 bp, Artificial Sequence
  synthetic oligonucleotide primer
cgagtggttttgaaacaggtttacaa

SEQ ID NO: 305 - DNA, 20 bp, Artificial Sequence
  synthetic oligonucleotide primer
cagcagagtgacccagtgat

SEQ ID NO: 306 - DNA, 23 bp, Artificial Sequence
  synthetic oligonucleotide primer
tgagtctcagaaaagaccccaca

SEQ ID NO: 307 - DNA, 24 bp, Artificial Sequence
  synthetic oligonucleotide primer
ctcctatactgactgggaggactt

SEQ ID NO: 308 - DNA, 19 bp, Artificial Sequence
  synthetic oligonucleotide primer
aatcagcacggagggtgag

SEQ ID NO: 309 - DNA, 18 bp, Artificial Sequence
  synthetic oligonucleotide primer
ccctcgctgactgttgct

SEQ ID NO: 310 - DNA, 21 bp, Artificial Sequence
  synthetic oligonucleotide primer
tgttcccacgtaacacacagg

SEQ ID NO: 311 - DNA, 21 bp, Artificial Sequence
  synthetic oligonucleotide primer
catggtgcaatctcttggcat

SEQ ID NO: 312 - DNA, 26 bp, Artificial Sequence
  synthetic oligonucleotide primer
tgagaagtcacctaccttgatgatga

SEQ ID NO: 313 - DNA, 25 bp, Artificial Sequence
  synthetic oligonucleotide primer
tgcaaaagctctaacttgtgtcctt

SEQ ID NO: 314 - DNA, 22 bp, Artificial Sequence
  synthetic oligonucleotide primer
gtaggtcttctgatgccagctc

SEQ ID NO: 315 - DNA, 19 bp, Artificial Sequence
  synthetic oligonucleotide primer
gggccaaagctttctgagg

SEQ ID NO: 316 - DNA, 20 bp, Artificial Sequence
  synthetic oligonucleotide primer
ctggtcgcggatcttcttct

SEQ ID NO: 317 - DNA, 21 bp, Artificial Sequence
  synthetic oligonucleotide primer
tctgttcccacccctacactt

SEQ ID NO: 318 - DNA, 22 bp, Artificial Sequence
  synthetic oligonucleotide primer
ctctgtccttgccagaagatgg

SEQ ID NO: 319 - DNA, 17 bp, Artificial Sequence
  synthetic oligonucleotide primer
gcccgggtggtctggat

SEQ ID NO: 320 - DNA, 17 bp, Artificial Sequence
  synthetic oligonucleotide primer
cccggcctccatctcct

SEQ ID NO: 321 - DNA, 22 bp, Artificial Sequence
  synthetic oligonucleotide primer
ctcccaggtcatcttctgcaat

SEQ ID NO: 322 - DNA, 18 bp, Artificial Sequence
  synthetic oligonucleotide primer
gggcttcagaccgtgcta

SEQ ID NO: 323 - DNA, 19 bp, Artificial Sequence
  synthetic oligonucleotide primer
ccaccggtgtggctcttta

SEQ ID NO: 324 - DNA, 27 bp, Artificial Sequence
  synthetic oligonucleotide primer
ctatcctgtacttaccacaacaacctt

SEQ ID NO: 325 - DNA, 21 bp, Artificial Sequence
  synthetic oligonucleotide primer
ccctagtctgccactgaggat

SEQ ID NO: 326 - DNA, 24 bp, Artificial Sequence
  synthetic oligonucleotide primer
cttcaatctcccatccgttgatgt

SEQ ID NO: 327 - DNA, 25 bp, Artificial Sequence
  synthetic oligonucleotide primer
gagtttgttatcattgcttggctca

SEQ ID NO: 328 - DNA, 22 bp, Artificial Sequence
  synthetic oligonucleotide primer
ttccatgaagcgcacaaacatc

SEQ ID NO: 329 - DNA, 28 bp, Artificial Sequence
  synthetic oligonucleotide primer
gagaactgatagaaattggatgtgagga

SEQ ID NO: 330 - DNA, 23 bp, Artificial Sequence
  synthetic oligonucleotide primer
cctgtgagtggatttcccatgtg

SEQ ID NO: 331 - DNA, 24 bp, Artificial Sequence
  synthetic oligonucleotide primer
gggagatggttaaatccacaacaa

SEQ ID NO: 332 - DNA, 22 bp, Artificial Sequence
  synthetic oligonucleotide primer
catctcctcatcttgctgccta

SEQ ID NO: 333 - DNA, 25 bp, Artificial Sequence
  synthetic oligonucleotide primer
ctccttcatgttcttgcttcttcct

SEQ ID NO: 334 - DNA, 28 bp, Artificial Sequence
  synthetic oligonucleotide primer
acaacagaagtataagaatggctgtcac

SEQ ID NO: 335 - DNA, 26 bp, Artificial Sequence
  synthetic oligonucleotide primer
ggtattgaatttctttggaccaggtg

SEQ ID NO: 336 - DNA, 27 bp, Artificial Sequence
  synthetic oligonucleotide primer
aaagattgtatgaggtcctgtcctagt

SEQ ID NO: 337 - DNA, 27 bp, Artificial Sequence
  synthetic oligonucleotide primer
catacaactgttttgaaaatccagcgt

SEQ ID NO: 338 - DNA, 23 bp, Artificial Sequence
  synthetic oligonucleotide primer
cctgacaagtaagcagggagaga

SEQ ID NO: 339 - DNA, 28 bp, Artificial Sequence
  synthetic oligonucleotide primer
ttatagctgatttgatggagttggacat

SEQ ID NO: 340 - DNA, 27 bp, Artificial Sequence
  synthetic oligonucleotide primer
ttcttgagtgaaggactgagaaaatcc

SEQ ID NO: 341 - DNA, 23 bp, Artificial Sequence
  synthetic oligonucleotide primer
gctgaactgtggatagtgagtgt

SEQ ID NO: 342 - DNA, 26 bp, Artificial Sequence
  synthetic oligonucleotide primer
caagtttacaactgcatgtttcagca

SEQ ID NO: 343 - DNA, 21 bp, Artificial Sequence
  synthetic oligonucleotide primer
tggcaagctggctgaaattct

SEQ ID NO: 344 - DNA, 22 bp, Artificial Sequence
  synthetic oligonucleotide primer
agacagatagcaccttcagcac

SEQ ID NO: 345 - DNA, 26 bp, Artificial Sequence
  synthetic oligonucleotide primer
ttcttccttctgtttttcaggctact

SEQ ID NO: 346 - DNA, 28 bp, Artificial Sequence
  synthetic oligonucleotide primer
cattccttttagatagccaggtatcact

SEQ ID NO: 347 - DNA, 28 bp, Artificial Sequence
  synthetic oligonucleotide primer
tttcgtaagtgttactcaagaagcagaa

SEQ ID NO: 348 - DNA, 26 bp, Artificial Sequence
  synthetic oligonucleotide primer
aagggacaacagttaagctttatggt

SEQ ID NO: 349 - DNA, 21 bp, Artificial Sequence
  synthetic oligonucleotide primer
ccagacgcatttccacagcta

SEQ ID NO: 350 - DNA, 28 bp, Artificial Sequence
  synthetic oligonucleotide primer
aggtcaacagattactgtatagtgcaag

SEQ ID NO: 351 - DNA, 27 bp, Artificial Sequence
  synthetic oligonucleotide primer
tttacataggtggaatgaatggctgaa

SEQ ID NO: 352 - DNA, 28 bp, Artificial Sequence
  synthetic oligonucleotide primer
agcggtataatcaggagtttttaaaggt

SEQ ID NO: 353 - DNA, 29 bp, Artificial Sequence
  synthetic oligonucleotide primer
cacagacactctagtatctggaaaaatgg

SEQ ID NO: 354 - DNA, 25 bp, Artificial Sequence
  synthetic oligonucleotide primer
agcatggagtttcctaagagatgga

SEQ ID NO: 355 - DNA, 26 bp, Artificial Sequence
  synthetic oligonucleotide primer
ctgtaaatcatctgtgaatccagagg

SEQ ID NO: 356 - DNA, 25 bp, Artificial Sequence
  synthetic oligonucleotide primer
agcacttacctgtgactccatagaa

SEQ ID NO: 357 - DNA, 27 bp, Artificial Sequence
  synthetic oligonucleotide primer
cacgattcttttagatctgagatgcac

SEQ ID NO: 358 - DNA, 27 bp, Artificial Sequence
  synthetic oligonucleotide primer
gtctcaaacacaaactagagtcacaca

SEQ ID NO: 359 - DNA, 22 bp, Artificial Sequence
  synthetic oligonucleotide primer
aaatggaaacttgcaccctgtt

SEQ ID NO: 360 - DNA, 26 bp, Artificial Sequence
  synthetic oligonucleotide primer
agagaaaaccattacttgtccatcgt

SEQ ID NO: 361 - DNA, 22 bp, Artificial Sequence
  synthetic oligonucleotide primer
tttgctccaaactgaccaaact

SEQ ID NO: 362 - DNA, 26 bp, Artificial Sequence
  synthetic oligonucleotide primer
ttcatgaaatactccaaagcctcttg

SEQ ID NO: 363 - DNA, 17 bp, Artificial Sequence
  synthetic oligonucleotide primer
agcgcccgcatgtacaa

SEQ ID NO: 364 - DNA, 18 bp, Artificial Sequence
  synthetic oligonucleotide primer
gggttctcctgggccatc

SEQ ID NO: 365 - DNA, 22 bp, Artificial Sequence
  synthetic oligonucleotide primer
agaaccccaagatgcacaactc

SEQ ID NO: 366 - DNA, 18 bp, Artificial Sequence
  synthetic oligonucleotide primer
ccgggcagcgtgtactta

SEQ ID NO: 367 - DNA, 18 bp, Artificial Sequence
  synthetic oligonucleotide primer
cgtgaaccagcgcatgga

SEQ ID NO: 368 - DNA, 21 bp, Artificial Sequence
  synthetic oligonucleotide primer
cgagccgttcatgtaggtctg

SEQ ID NO: 369 - DNA, 18 bp, Artificial Sequence
  synthetic oligonucleotide primer
cccctggcatggctcttg

SEQ ID NO: 370 - DNA, 17 bp, Artificial Sequence
  synthetic oligonucleotide primer
gggccgctctggtagtg

SEQ ID NO: 371 - DNA, 17 bp, Artificial Sequence
  synthetic oligonucleotide primer
ggcccctgagcgtcatc

SEQ ID NO: 372 - DNA, 19 bp, Artificial Sequence
  synthetic oligonucleotide primer
gcacggtaacgtagggtgt

SEQ ID NO: 373 - DNA, 17 bp, Artificial Sequence
  synthetic oligonucleotide primer
ggcctcaacgcccatgt

SEQ ID NO: 374 - DNA, 18 bp, Artificial Sequence
  synthetic oligonucleotide primer
cgggaagcgggagatctt

SEQ ID NO: 375 - DNA, 20 bp, Artificial Sequence
  synthetic oligonucleotide primer
ggagaggtggagaggcttca

SEQ ID NO: 376 - DNA, 19 bp, Artificial Sequence
  synthetic oligonucleotide primer
gcgtcctactggcatgacc

SEQ ID NO: 377 - DNA, 19 bp, Artificial Sequence
  synthetic oligonucleotide primer
gacagcctgacctcacctt

SEQ ID NO: 378 - DNA, 18 bp, Artificial Sequence
  synthetic oligonucleotide primer
cctgaggacccagtggag

SEQ ID NO: 379 - DNA, 17 bp, Artificial Sequence
  synthetic oligonucleotide primer
acctgtcggcgcctttc

SEQ ID NO: 380 - DNA, 17 bp, Artificial Sequence
  synthetic oligonucleotide primer
ggagtggctgtgcacca

SEQ ID NO: 381 - DNA, 26 bp, Artificial Sequence
  synthetic oligonucleotide primer
acggattatttagtcatcgtggagga

SEQ ID NO: 382 - DNA, 27 bp, Artificial Sequence
  synthetic oligonucleotide primer
aacattaaatgggatggtctggaactt

SEQ ID NO: 383 - DNA, 20 bp, Artificial Sequence
  synthetic oligonucleotide primer
ggtgcactgggactttggta

SEQ ID NO: 384 - DNA, 22 bp, Artificial Sequence
  synthetic oligonucleotide primer
gaaaagggagtcttgggaggtt

SEQ ID NO: 385 - DNA, 25 bp, Artificial Sequence
  synthetic oligonucleotide primer
ttttctgagaacaggaagttggtag

SEQ ID NO: 386 - DNA, 23 bp, Artificial Sequence
  synthetic oligonucleotide primer
acaaccacatgtgtccagtgaaa

SEQ ID NO: 387 - DNA, 23 bp, Artificial Sequence
  synthetic oligonucleotide primer
catacccatctcctaacggcttt

SEQ ID NO: 388 - DNA, 22 bp, Artificial Sequence
  synthetic oligonucleotide primer
gcaacatctctctttgcaccca

SEQ ID NO: 389 - DNA, 24 bp, Artificial Sequence
  synthetic oligonucleotide primer
gctattcagctacagatggcttga

SEQ ID NO: 390 - DNA, 21 bp, Artificial Sequence
  synthetic oligonucleotide primer
gtgaaggaggatgagcctgac

SEQ ID NO: 391 - DNA, 28 bp, Artificial Sequence
  synthetic oligonucleotide primer
caaggaagaagatcatactcaacacgat

SEQ ID NO: 392 - DNA, 28 bp, Artificial Sequence
  synthetic oligonucleotide primer
tccattcattctgcttattctcattcgt

SEQ ID NO: 393 - DNA, 21 bp, Artificial Sequence
  synthetic oligonucleotide primer
ccagtggatgtgcagacacta

SEQ ID NO: 394 - DNA, 27 bp, Artificial Sequence
  synthetic oligonucleotide primer
agcctaaacatccccttaaattggatt

SEQ ID NO: 395 - DNA, 23 bp, Artificial Sequence
  synthetic oligonucleotide primer
ccagagtgctctaatgactgaga

SEQ ID NO: 396 - DNA, 20 bp, Artificial Sequence
  synthetic oligonucleotide primer
tgacatggaaagcccctgtt

SEQ ID NO: 397 - DNA, 20 bp, Artificial Sequence
  synthetic oligonucleotide primer
gtactgcatgcgcttgacat

SEQ ID NO: 398 - DNA, 28 bp, Artificial Sequence
  synthetic oligonucleotide primer
ttgataacctgacagacaataaaaggca

SEQ ID NO: 399 - DNA, 20 bp, Artificial Sequence
  synthetic oligonucleotide primer
ccatgaccacccttgggtat

SEQ ID NO: 400 - DNA, 28 bp, Artificial Sequence
  synthetic oligonucleotide primer
tgcagaagattcttataaagtgcagctt

SEQ ID NO: 401 - DNA, 22 bp, Artificial Sequence
  synthetic oligonucleotide primer
gggatgaggaggtagagcatga

SEQ ID NO: 402 - DNA, 27 bp, Artificial Sequence
  synthetic oligonucleotide primer
ctctctgtaaagttactcttggttgct

SEQ ID NO: 403 - DNA, 27 bp, Artificial Sequence
  synthetic oligonucleotide primer
taaatggttttcttttctcctccaacc

SEQ ID NO: 404 - DNA, 22 bp, Artificial Sequence
  synthetic oligonucleotide primer
gcaggactgtcaagcagagaat

SEQ ID NO: 405 - DNA, 27 bp, Artificial Sequence
  synthetic oligonucleotide primer
tctgttcaattttgttgagcttctgaa

SEQ ID NO: 406 - DNA, 28 bp, Artificial Sequence
  synthetic oligonucleotide primer
aagatgctctgagtctaatgaagttgtc

SEQ ID NO: 407 - DNA, 28 bp, Artificial Sequence
  synthetic oligonucleotide primer
gaaagaacaacacttgaaaatctgagca

SEQ ID NO: 408 - DNA, 24 bp, Artificial Sequence
  synthetic oligonucleotide primer
gatatcactccgatgacacagaca

SEQ ID NO: 409 - DNA, 20 bp, Artificial Sequence
  synthetic oligonucleotide primer
ccgagatggccttgaagtca

SEQ ID NO: 410 - DNA, 26 bp, Artificial Sequence
  synthetic oligonucleotide primer
ctgtgtttattgtttcaggatggcaa

SEQ ID NO: 411 - DNA, 22 bp, Artificial Sequence
  synthetic oligonucleotide primer
ggcagtaggaaagtccttgaca

SEQ ID NO: 412 - DNA, 24 bp, Artificial Sequence
  synthetic oligonucleotide primer
agcattcaggaagaaagaggcatt

SEQ ID NO: 413 - DNA, 22 bp, Artificial Sequence
  synthetic oligonucleotide primer
ccatagcatgcaggaagcacta

SEQ ID NO: 414 - DNA, 25 bp, Artificial Sequence
  synthetic oligonucleotide primer
caaggagtttgtttgttcctttgct

SEQ ID NO: 415 - DNA, 25 bp, Artificial Sequence
  synthetic oligonucleotide primer
agagaggcatgttaaaattgggtga

SEQ ID NO: 416 - DNA, 28 bp, Artificial Sequence
  synthetic oligonucleotide primer
cccaattattgaaggaaatgtccatacc

SEQ ID NO: 417 - DNA, 27 bp, Artificial Sequence
  synthetic oligonucleotide primer
cttcaattttatttcctccctggaagt

SEQ ID NO: 418 - DNA, 23 bp, Artificial Sequence
  synthetic oligonucleotide primer
aggctgcgttggaagttatttct

SEQ ID NO: 419 - DNA, 22 bp, Artificial Sequence
  synthetic oligonucleotide primer
ggaccctgacaaatgtgctgtt

SEQ ID NO: 420 - DNA, 27 bp, Artificial Sequence
  synthetic oligonucleotide primer
tgttatatgctgtgctttggaagttca

SEQ ID NO: 421 - DNA, 26 bp, Artificial Sequence
  synthetic oligonucleotide primer
agtatttggaggtctggctttgaatc

SEQ ID NO: 422 - DNA, 28 bp, Artificial Sequence
  synthetic oligonucleotide primer
gcttatttgctctctcatgttctgtttt

SEQ ID NO: 423 - DNA, 25 bp, Artificial Sequence
  synthetic oligonucleotide primer
gctcttcacttcatgtccacatcaa

SEQ ID NO: 424 - DNA, 28 bp, Artificial Sequence
  synthetic oligonucleotide primer
ctttgtaattaccagctcagatgatgga

SEQ ID NO: 425 - DNA, 25 bp, Artificial Sequence
  synthetic oligonucleotide primer
gaatctgcattcccagagacaagaa

SEQ ID NO: 426 - DNA, 28 bp, Artificial Sequence
  synthetic oligonucleotide primer
ggtcagtaattgataggaagagtatcca

SEQ ID NO: 427 - DNA, 21 bp, Artificial Sequence
  synthetic oligonucleotide primer
ctaacaaccctcctgccatca

SEQ ID NO: 428 - DNA, 28 bp, Artificial Sequence
  synthetic oligonucleotide primer
ccttgactaaatctaccatgttttctca

SEQ ID NO: 429 - DNA, 27 bp, Artificial Sequence
  synthetic oligonucleotide primer
caataatagaggaagaagtcccaacca

SEQ ID NO: 430 - DNA, 26 bp, Artificial Sequence
  synthetic oligonucleotide primer
ctgagaacattagtgggacatacagg

SEQ ID NO: 431 - DNA, 22 bp, Artificial Sequence
  synthetic oligonucleotide primer
ttactttgcctgtgactgctga

SEQ ID NO: 432 - DNA, 28 bp, Artificial Sequence
  synthetic oligonucleotide primer
ctgttcctgtttatgccttcatttttct

SEQ ID NO: 433 - DNA, 28 bp, Artificial Sequence
  synthetic oligonucleotide primer
ggtactaacactgattaacggtttctgt

SEQ ID NO: 434 - DNA, 25 bp, Artificial Sequence
  synthetic oligonucleotide primer
ggtgaaggcaatttactcttgaact

SEQ ID NO: 435 - DNA, 28 bp, Artificial Sequence
  synthetic oligonucleotide primer
cttgttttcagaatcactctgcttttca

SEQ ID NO: 436 - DNA, 26 bp, Artificial Sequence
  synthetic oligonucleotide primer
tttcttagttggcactctatgtgctt

SEQ ID NO: 437 - DNA, 27 bp, Artificial Sequence
  synthetic oligonucleotide primer
acctctttagggagcaatgaaatgaag

SEQ ID NO: 438 - DNA, 29 bp, Artificial Sequence
  synthetic oligonucleotide primer
cctgtaatttgggacatctgttaaaacaa

SEQ ID NO: 439 - DNA, 28 bp, Artificial Sequence
  synthetic oligonucleotide primer
ttattttgcagcaattaagtgaggcatt

SEQ ID NO: 440 - DNA, 23 bp, Artificial Sequence
  synthetic oligonucleotide primer
gcttgtaccatgttcagcaacac

SEQ ID NO: 441 - DNA, 25 bp, Artificial Sequence
  synthetic oligonucleotide primer
ctgagactttgcatggtttctttcc

SEQ ID NO: 442 - DNA, 28 bp, Artificial Sequence
  synthetic oligonucleotide primer
aaccatgctgactcaagatttgatagtt

SEQ ID NO: 443 - DNA, 23 bp, Artificial Sequence
  synthetic oligonucleotide primer
acaacttcaccattccttgcagt

SEQ ID NO: 444 - DNA, 24 bp, Artificial Sequence
  synthetic oligonucleotide primer
tgatagtctagccaaggtccaaga

SEQ ID NO: 445 - DNA, 21 bp, Artificial Sequence
  synthetic oligonucleotide primer
agagagaacgcggaattggtc

SEQ ID NO: 446 - DNA, 28 bp, Artificial Sequence
  synthetic oligonucleotide primer
gcttcttctaagtgcatttctctcatct

SEQ ID NO: 447 - DNA, 25 bp, Artificial Sequence
  synthetic oligonucleotide primer
ctgagagcactgatgataaacacct

SEQ ID NO: 448 - DNA, 25 bp, Artificial Sequence
  synthetic oligonucleotide primer
gttcttcttcagagtaacgttcact

SEQ ID NO: 449 - DNA, 28 bp, Artificial Sequence
  synthetic oligonucleotide primer
acagacttattgtgtagaagatactcca

SEQ ID NO: 450 - DNA, 23 bp, Artificial Sequence
  synthetic oligonucleotide primer
gatttggttctagggtgctgtga

SEQ ID NO: 451 - DNA, 19 bp, Artificial Sequence
  synthetic oligonucleotide primer
cgattgccagctccgttca

SEQ ID NO: 452 - DNA, 23 bp, Artificial Sequence
  synthetic oligonucleotide primer
gcatttactgcagcttgcttagg

SEQ ID NO: 453 - DNA, 24 bp, Artificial Sequence
  synthetic oligonucleotide primer
aatgcctccagttcaggaaaatga

SEQ ID NO: 454 - DNA, 19 bp, Artificial Sequence
  synthetic oligonucleotide primer
gcagtctgggctggctttt

SEQ ID NO: 455 - DNA, 22 bp, Artificial Sequence
  synthetic oligonucleotide primer
ggaggagttgaagtttgtggga

SEQ ID NO: 456 - DNA, 21 bp, Artificial Sequence
  synthetic oligonucleotide primer
cccaccctcaggactatacca

SEQ ID NO: 457 - DNA, 19 bp, Artificial Sequence
  synthetic oligonucleotide primer
ccagcccaacgtgctttac

SEQ ID NO: 458 - DNA, 20 bp, Artificial Sequence
  synthetic oligonucleotide primer
gtgtgttaatggcccctgga

SEQ ID NO: 459 - DNA, 19 bp, Artificial Sequence
  synthetic oligonucleotide primer
ccaggtgagggaggtgagt

SEQ ID NO: 460 - DNA, 23 bp, Artificial Sequence
  synthetic oligonucleotide primer
ggaagtaggtactgggagattgg

SEQ ID NO: 461 - DNA, 21 bp, Artificial Sequence
  synthetic oligonucleotide primer
gcattagcaagcttgggctca

SEQ ID NO: 462 - DNA, 24 bp, Artificial Sequence
  synthetic oligonucleotide primer
gacatcttcccactaatgccagat

SEQ ID NO: 463 - DNA, 18 bp, Artificial Sequence
  synthetic oligonucleotide primer
tcaggcctcttgggagga

SEQ ID NO: 464 - DNA, 18 bp, Artificial Sequence
  synthetic oligonucleotide primer
gcgatgtgtgggcaatgg

SEQ ID NO: 465 - DNA, 25 bp, Artificial Sequence
  synthetic oligonucleotide primer
tgacctttttggttacccacactta

SEQ ID NO: 466 - DNA, 28 bp, Artificial Sequence
  synthetic oligonucleotide primer
aactaaaaggctgaaatcaagtagggtt

SEQ ID NO: 467 - DNA, 25 bp, Artificial Sequence
  synthetic oligonucleotide primer
gggtgtaaaataggtggaactcaaa

SEQ ID NO: 468 - DNA, 28 bp, Artificial Sequence
  synthetic oligonucleotide primer
acagaattaaaccttgacacaacatcca

SEQ ID NO: 469 - DNA, 27 bp, Artificial Sequence
  synthetic oligonucleotide primer
ttttcagggacaagaatccttcaagaa

SEQ ID NO: 470 - DNA, 26 bp, Artificial Sequence
  synthetic oligonucleotide primer
acattcaagtctatgcaaaccagaca

SEQ ID NO: 471 - DNA, 28 bp, Artificial Sequence
  synthetic oligonucleotide primer
ggtatctctctcggtgtatttctctact

SEQ ID NO: 472 - DNA, 28 bp, Artificial Sequence
  synthetic oligonucleotide primer
acacttaaaaagggtaaaggcagaatca

SEQ ID NO: 473 - DNA, 27 bp, Artificial Sequence
  synthetic oligonucleotide primer
agatgttgaactatgcaaagagacatt

SEQ ID NO: 474 - DNA, 27 bp, Artificial Sequence
  synthetic oligonucleotide primer
tctgcattataaaaaggacagccagat

SEQ ID NO: 475 - DNA, 29 bp, Artificial Sequence
  synthetic oligonucleotide primer
gggccttagtgttcttttgtaattaatga

SEQ ID NO: 476 - DNA, 22 bp, Artificial Sequence
  synthetic oligonucleotide primer
aaccatgccaaatgtggaaacc

SEQ ID NO: 477 - DNA, 28 bp, Artificial Sequence
  synthetic oligonucleotide primer
cttgtgattgactttaaacttgttggca

SEQ ID NO: 478 - DNA, 28 bp, Artificial Sequence
  synthetic oligonucleotide primer
ccaactaccttgtaacagaaaagctaac

SEQ ID NO: 479 - DNA, 26 bp, Artificial Sequence
  synthetic oligonucleotide primer
agttaagctggattgtttttcctctt

SEQ ID NO: 480 - DNA, 24 bp, Artificial Sequence
  synthetic oligonucleotide primer
actcctccaaaaaggcttcaatca

SEQ ID NO: 481 - DNA, 25 bp, Artificial Sequence
  synthetic oligonucleotide primer
actcgtctcctctatggatttgact

SEQ ID NO: 482 - DNA, 21 bp, Artificial Sequence
  synthetic oligonucleotide primer
gggacactatacaagggcaca

SEQ ID NO: 483 - DNA, 26 bp, Artificial Sequence
  synthetic oligonucleotide primer
gttcctcaaaagagaaatcacgcatt

SEQ ID NO: 484 - DNA, 24 bp, Artificial Sequence
  synthetic oligonucleotide primer
gtaaatttctcatgggcagctcct

SEQ ID NO: 485 - DNA, 18 bp, Artificial Sequence
  synthetic oligonucleotide primer
cacgtgcaaggacacctg

SEQ ID NO: 486 - DNA, 26 bp, Artificial Sequence
  synthetic oligonucleotide primer
ccaaagactctccaagatgggatact

SEQ ID NO: 487 - DNA, 26 bp, Artificial Sequence
  synthetic oligonucleotide primer
catgcatgaacatttttctccacctt

SEQ ID NO: 488 - DNA, 27 bp, Artificial Sequence
  synthetic oligonucleotide primer
ggaaatgttctgttctccttcactttc

SEQ ID NO: 489 - DNA, 21 bp, Artificial Sequence
  synthetic oligonucleotide primer
tgaggtgacccttgtctctgt

SEQ ID NO: 490 - DNA, 19 bp, Artificial Sequence
  synthetic oligonucleotide primer
ctccccaccagaccatgag

SEQ ID NO: 491 - DNA, 22 bp, Artificial Sequence
  synthetic oligonucleotide primer
gcaccatctcacaattgccagt

SEQ ID NO: 492 - DNA, 22 bp, Artificial Sequence
  synthetic oligonucleotide primer
agctgccagacatgagaaaagg

SEQ ID NO: 493 - DNA, 19 bp, Artificial Sequence
  synthetic oligonucleotide primer
ccacactgacgtgcctctc

SEQ ID NO: 494 - DNA, 20 bp, Artificial Sequence
  synthetic oligonucleotide primer
acctttgcgatctgcacaca

SEQ ID NO: 495 - DNA, 21 bp, Artificial Sequence
  synthetic oligonucleotide primer
cctcacagcagggtcttctct

SEQ ID NO: 496 - DNA, 20 bp, Artificial Sequence
  synthetic oligonucleotide primer
tcaggaaaatgctggctgac

SEQ ID NO: 497 - DNA, 24 bp, Artificial Sequence
  synthetic oligonucleotide primer
attcatgatcccactgccttcttt

SEQ ID NO: 498 - DNA, 20 bp, Artificial Sequence
  synthetic oligonucleotide primer
gctaggcagtgtggacagac

SEQ ID NO: 499 - DNA, 20 bp, Artificial Sequence
  synthetic oligonucleotide primer
ctcattagctgtggcagcgt

SEQ ID NO: 500 - DNA, 28 bp, Artificial Sequence
  synthetic oligonucleotide primer
tggtattgcctacaaagaagttgatgaa

SEQ ID NO: 501 - DNA, 21 bp, Artificial Sequence
  synthetic oligonucleotide primer
ggcccagcttgctagacaaat

SEQ ID NO: 502 - DNA, 24 bp, Artificial Sequence
  synthetic oligonucleotide primer
gtccgtaaaaatgctggagacatc

SEQ ID NO: 503 - DNA, 28 bp, Artificial Sequence
  synthetic oligonucleotide primer
agttctatgttgtccttgtaggttttcc

SEQ ID NO: 504 - DNA, 24 bp, Artificial Sequence
  synthetic oligonucleotide primer
cccagcaaagcattttaagatcct

SEQ ID NO: 505 - DNA, 25 bp, Artificial Sequence
  synthetic oligonucleotide primer
ctctctgttttaagatctgggcagt

SEQ ID NO: 506 - DNA, 27 bp, Artificial Sequence
  synthetic oligonucleotide primer
acaacccactgaggtatatgtataggt

SEQ ID NO: 507 - DNA, 23 bp, Artificial Sequence
  synthetic oligonucleotide primer
gttacgcagtgctaaccaagttc

SEQ ID NO: 508 - DNA, 26 bp, Artificial Sequence
  synthetic oligonucleotide primer
gttgcaaaccacaaaagtatactcca

SEQ ID NO: 509 - DNA, 23 bp, Artificial Sequence
  synthetic oligonucleotide primer
gtcctttctgtaggctggatgaa

SEQ ID NO: 510 - DNA, 25 bp, Artificial Sequence
  synthetic oligonucleotide primer
aagaggagaaactcagagataacca

SEQ ID NO: 511 - DNA, 20 bp, Artificial Sequence
  synthetic oligonucleotide primer
gctgccttgacaccgtcttt

SEQ ID NO: 512 - DNA, 18 bp, Artificial Sequence
  synthetic oligonucleotide primer
ccgaaggccgcgatgtag

SEQ ID NO: 513 - DNA, 20 bp, Artificial Sequence
  synthetic oligonucleotide primer
ctcacctctctgcacagctc

SEQ ID NO: 514 - DNA, 22 bp, Artificial Sequence
  synthetic oligonucleotide primer
ggattgccacagtgaggacaaa

SEQ ID NO: 515 - DNA, 20 bp, Artificial Sequence
  synthetic oligonucleotide primer
ggccagtaacccaccttctg

SEQ ID NO: 516 - DNA, 24 bp, Artificial Sequence
  synthetic oligonucleotide primer
cccagtatattttgttgcccaact

SEQ ID NO: 517 - DNA, 22 bp, Artificial Sequence
  synthetic oligonucleotide primer
gaaagcctcacctgtctacgtt

SEQ ID NO: 518 - DNA, 17 bp, Artificial Sequence
  synthetic oligonucleotide primer
cccacctgcaccaggta

SEQ ID NO: 519 - DNA, 23 bp, Artificial Sequence
  synthetic oligonucleotide primer
agcggatcaagaagagcaagatg

SEQ ID NO: 520 - DNA, 26 bp, Artificial Sequence
  synthetic oligonucleotide primer
agtcggtcttccaaataatctgtgtg

SEQ ID NO: 521 - DNA, 20 bp, Artificial Sequence
  synthetic oligonucleotide primer
agggaccgggaagtcactat

SEQ ID NO: 522 - DNA, 21 bp, Artificial Sequence
  synthetic oligonucleotide primer
caccttcctccagaagcttga

SEQ ID NO: 523 - DNA, 21 bp, Artificial Sequence
  synthetic oligonucleotide primer
cctggtccctgttgttgatgt

SEQ ID NO: 524 - DNA, 29 bp, Artificial Sequence
  synthetic oligonucleotide primer
gcattgctctaggaattatagtaggttgt

SEQ ID NO: 525 - DNA, 22 bp, Artificial Sequence
  synthetic oligonucleotide primer
cagcatctcagggccaaaaatt

SEQ ID NO: 526 - DNA, 27 bp, Artificial Sequence
  synthetic oligonucleotide primer
gctctgataggaaaatgagatctactg

SEQ ID NO: 527 - DNA, 24 bp, Artificial Sequence
  synthetic oligonucleotide primer
aagctcacctgagtactcctactt

SEQ ID NO: 528 - DNA, 28 bp, Artificial Sequence
  synthetic oligonucleotide primer
atggagttagggctatgataattagtga

SEQ ID NO: 529 - DNA, 24 bp, Artificial Sequence
  synthetic oligonucleotide primer
tgacttgtcacaatgtcaccacat

SEQ ID NO: 530 - DNA, 27 bp, Artificial Sequence
  synthetic oligonucleotide primer
tttttctgtttggcttgacttgacttt

SEQ ID NO: 531 - DNA, 24 bp, Artificial Sequence
  synthetic oligonucleotide primer
gatccaaaagaaagcggttcaagt

SEQ ID NO: 532 - DNA, 21 bp, Artificial Sequence
  synthetic oligonucleotide primer
gcttctgggttttgcacaagt

SEQ ID NO: 533 - DNA, 26 bp, Artificial Sequence
  synthetic oligonucleotide primer
ctaaaaaccctcctttgtccagagtt

SEQ ID NO: 534 - DNA, 21 bp, Artificial Sequence
  synthetic oligonucleotide primer
cccgtcttcatgctcactgac

SEQ ID NO: 535 - DNA, 28 bp, Artificial Sequence
  synthetic oligonucleotide primer
caactatcccagaagtattcaagtccat

SEQ ID NO: 536 - DNA, 24 bp, Artificial Sequence
  synthetic oligonucleotide primer
aactgaaattattcactgggctgt

SEQ ID NO: 537 - DNA, 19 bp, Artificial Sequence
  synthetic oligonucleotide primer
agaaggctgccacatgcaa

SEQ ID NO: 538 - DNA, 22 bp, Artificial Sequence
  synthetic oligonucleotide primer
cttttgtgtgttctgtcaggct

SEQ ID NO: 539 - DNA, 26 bp, Artificial Sequence
  synthetic oligonucleotide primer
acaatgccacctgaatacaggttatc

SEQ ID NO: 540 - DNA, 25 bp, Artificial Sequence
  synthetic oligonucleotide primer
gtctactttgtccccagtccatttt

SEQ ID NO: 541 - DNA, 21 bp, Artificial Sequence
  synthetic oligonucleotide primer
tttttggagccccgctgaata

SEQ ID NO: 542 - DNA, 25 bp, Artificial Sequence
  synthetic oligonucleotide primer
catacggtgatgagtgaagaacctc

SEQ ID NO: 543 - DNA, 28 bp, Artificial Sequence
  synthetic oligonucleotide primer
accaaagacctatttagttctcatgcaa

SEQ ID NO: 544 - DNA, 22 bp, Artificial Sequence
  synthetic oligonucleotide primer
tggattcattggcctgcatgat

SEQ ID NO: 545 - DNA, 22 bp, Artificial Sequence
  synthetic oligonucleotide primer
caccttcttggaggccagatac

SEQ ID NO: 546 - DNA, 17 bp, Artificial Sequence
  synthetic oligonucleotide primer
ggccccatggcctcttc

SEQ ID NO: 547 - DNA, 23 bp, Artificial Sequence
  synthetic oligonucleotide primer
gcaagcaaggaatgccttcaaaa

SEQ ID NO: 548 - DNA, 20 bp, Artificial Sequence
  synthetic oligonucleotide primer
gctctagggtgaccccactc

SEQ ID NO: 549 - DNA, 21 bp, Artificial Sequence
  synthetic oligonucleotide primer
gtggtgctgagtgtgcaaatc

SEQ ID NO: 550 - DNA, 19 bp, Artificial Sequence
  synthetic oligonucleotide primer
tgatgacctcgcccctgta

SEQ ID NO: 551 - DNA, 22 bp, Artificial Sequence
  synthetic oligonucleotide primer
ctggacataaggcaggttgtct

SEQ ID NO: 552 - DNA, 21 bp, Artificial Sequence
  synthetic oligonucleotide primer
gcaaggtccccatgacaagtg

SEQ ID NO: 553 - DNA, 21 bp, Artificial Sequence
  synthetic oligonucleotide primer
tcccctcttaaacccaatgcc

SEQ ID NO: 554 - DNA, 22 bp, Artificial Sequence
  synthetic oligonucleotide primer
actgcactagccttggtgaaat

SEQ ID NO: 555 - DNA, 25 bp, Artificial Sequence
  synthetic oligonucleotide primer
ccttctagtcttcagaacgaatggt

SEQ ID NO: 556 - DNA, 32 bp, Artificial Sequence
  synthetic oligonucleotide primer
tgtcacatgaatgtaaatcaagaaaacagatg

SEQ ID NO: 557 - DNA, 28 bp, Artificial Sequence
  synthetic oligonucleotide primer
tttctgaactatttatggacaacagtca

SEQ ID NO: 558 - DNA, 23 bp, Artificial Sequence
  synthetic oligonucleotide primer
aaacagatgctctgagaaaggca

SEQ ID NO: 559 - DNA, 25 bp, Artificial Sequence
  synthetic oligonucleotide primer
gggcttgaacatactaaatgctcca

SEQ ID NO: 560 - DNA, 27 bp, Artificial Sequence
  synthetic oligonucleotide primer
actgtcctttggcaaaactgtaatact

SEQ ID NO: 561 - DNA, 17 bp, Artificial Sequence
  synthetic oligonucleotide primer
ggcagttgtggccctgt

SEQ ID NO: 562 - DNA, 28 bp, Artificial Sequence
  synthetic oligonucleotide primer
gccattgcgagaactttatccataagta

SEQ ID NO: 563 - DNA, 19 bp, Artificial Sequence
  synthetic oligonucleotide primer
ctcccgggctgaactttct

SEQ ID NO: 564 - DNA, 17 bp, Artificial Sequence
  synthetic oligonucleotide primer
gtctgcccgtggacctg

SEQ ID NO: 565 - DNA, 17 bp, Artificial Sequence
  synthetic oligonucleotide primer
agcaccaccagcgtgtc

SEQ ID NO: 566 - DNA, 22 bp, Artificial Sequence
  synthetic oligonucleotide primer
acacaagcttcctttccgtcat

SEQ ID NO: 567 - DNA, 20 bp, Artificial Sequence
  synthetic oligonucleotide primer
cgaagcgctacctgattcca

SEQ ID NO: 568 - DNA, 18 bp, Artificial Sequence
  synthetic oligonucleotide primer
ggagcagcatggagcctt

SEQ ID NO: 569 - DNA, 24 bp, Artificial Sequence
  synthetic oligonucleotide primer
ttccccacaccctactttctatca

SEQ ID NO: 570 - DNA, 24 bp, Artificial Sequence
  synthetic oligonucleotide primer
tgttaaccttgcagaatggtcgat

SEQ ID NO: 571 - DNA, 19 bp, Artificial Sequence
  synthetic oligonucleotide primer
gctcatcaccacgctccat

SEQ ID NO: 572 - DNA, 17 bp, Artificial Sequence
  synthetic oligonucleotide primer
cggcagtcccagcctac

SEQ ID NO: 573 - DNA, 21 bp, Artificial Sequence
  synthetic oligonucleotide primer
gagtatgcgctgaagctccat

SEQ ID NO: 574 - DNA, 20 bp, Artificial Sequence
  synthetic oligonucleotide primer
cgcgagaccctctcttcaga

SEQ ID NO: 575 - DNA, 17 bp, Artificial Sequence
  synthetic oligonucleotide primer
gcggagccacgtgttga

SEQ ID NO: 576 - DNA, 27 bp, Artificial Sequence
  synthetic oligonucleotide primer
cctacctgtggatgaagtttttcttct

SEQ ID NO: 577 - DNA, 17 bp, Artificial Sequence
  synthetic oligonucleotide primer
gctgcccgaaactgcct

SEQ ID NO: 578 - DNA, 22 bp, Artificial Sequence
  synthetic oligonucleotide primer
agtcaggaaggaccacttcagt

SEQ ID NO: 579 - DNA, 17 bp, Artificial Sequence
  synthetic oligonucleotide primer
gcgcgccgtttacttga

SEQ ID NO: 580 - DNA, 18 bp, Artificial Sequence
  synthetic oligonucleotide primer
ccctcgcagcacagctac

SEQ ID NO: 581 - DNA, 17 bp, Artificial Sequence
  synthetic oligonucleotide primer
ccagtggctgcacgtct

SEQ ID NO: 582 - DNA, 21 bp, Artificial Sequence
  synthetic oligonucleotide primer
ttacagatgcagcagcagaac

SEQ ID NO: 583 - DNA, 18 bp, Artificial Sequence
  synthetic oligonucleotide primer
ccgccgcttcttcttgct

SEQ ID NO: 584 - DNA, 17 bp, Artificial Sequence
  synthetic oligonucleotide primer
tgatgtccgggcacctg

SEQ ID NO: 585 - DNA, 17 bp, Artificial Sequence
  synthetic oligonucleotide primer
tgcaggcagagcctgtt

SEQ ID NO: 586 - DNA, 17 bp, Artificial Sequence
  synthetic oligonucleotide primer
ggtcccagcccctctct

SEQ ID NO: 587 - DNA, 17 bp, Artificial Sequence
  synthetic oligonucleotide primer
agcgaggccttcacctg

SEQ ID NO: 588 - DNA, 20 bp, Artificial Sequence
  synthetic oligonucleotide primer
cgcaacagctccttccactt